(19) United States
(12) Patent Application Publication
(10) Pub. No.: US 2009/0111692 A1
(43) Pub. Date: Apr. 30, 2009

(54) 3-(Pyridin-2-Yl)-[1,2,4]-Triazines as Fungicides

(76) Inventors: Wassilios Grammenos, Ludwigshafen (DE); Thomas Grote, Wachenheim (DE); Jochen Dietz, Mannheim (DE); Jan Klaas Lohmann, Ludwigshafen (DE); Jens Renner, Bad Durkheim (DE); Bernd Muller, Frankenthal (DE); Sarah Ulmschneider, Bad Durkheim (DE)

Correspondence Address: BIRCH STEWART KOLASCH & BIRCH, PO BOX 747, FALLS CHURCH, VA, 22040-0747, US

(21) Appl. No.: 12/226,026

(22) PCT Filed: Apr. 11, 2007
(86) PCT No.: PCT/EP2007/053515

§ 371 Date: Oct. 6, 2008

(30) Foreign Application Priority Data
Apr. 12, 2006 (EP) 06007743.5

(51) Int. Cl. A01N 43/707; C07D 401/06; A01P 3/00

(52) U.S. Cl. 504/100; 544/182; 544/183; 514/242; 514/243

(57) ABSTRACT

The invention relates to 3-(pyridin-2-yl)-[1,2,4]-triazines of formula (I) and their use in the control of parasitic fungi and to herbicides that contain said compounds as an effective ingredient thereof. In formula (I), R¹, R² independently represent OH, halogen, NO₂, NH₂, C₁-C₈ alkyl, C₁-C₈ alkoxy, C₁-C₈ halogenalkyl, C₁-C₈ halogenalkoxy, C₁-C₈ alkylamino or di(C₁-C₈ alkyl)amino, or they form, together with the C atoms to which they are bound, a saturated five-, six- or seven-membered carbocycle or heterocycle, which, in addition to the carbon ring members, has one or two heteroatoms selected from oxygen or sulfur as the ring members, the carbocycle and the heterocycle being unsubstituted or having 1, 2, 3 or 4 C₁-C₄ alkyl groups as the substituents; R³ represents hydrogen, C₁-C₄ alkyl, C₁-C₄ alkoxy, C₁-C₄ halogenalkyl, C₁-C₄ halogenalkoxy, C₃-C₆ cycloalkyl, C₃-C₆ cycloalkylmethyl, or halogen; R⁴ represents hydrogen, C₁-C₄ alkyl, C₁-C₄ alkoxy, C₁-C₄ halogenalkyl, C₁-C₄ halogenalkoxy or halogen; R⁵ represents C₁-C₈ alkyl, C₁-C₈ halogenalkyl, C₁-C₈ alkoxy, C₁-C₈ halogenalkoxy, C₃-C₈ cycloalkyl, C₃-C₈ cycloalkyloxy, five- or six-membered heteroaryl, phenyl, phenoxy, benzyl, benzyloxy, five- or six-membered heteroarylmethyl or five- or six-membered heteroaryloxy, said cyclic groups being unsubstituted or having 1, 2, 3, 4 or 5 groups R^{a}.

The present invention relates to 3-(pyridin-2-yl)-[1,2,4]-triazines and their use for controlling harmful fungi, and also to crop protection compositions comprising such compounds as active component.

EP-A 234 104 describes 2-(pyridin-2-yl)pyrimidines which have an alkyl group in the 6-position of the pyridine radical and which may have a fused saturated 5- or 6-membered ring in the 3,4-position of the pyrimidine ring. The compounds are suitable for controlling phytopathogenic fungi (harmful fungi).

2-(Pyridin-2-yl)pyrimidines having fungicidal action which carry an optionally substituted phenyl ring in the 4-position of the pyrimidine ring are known from U.S. Pat. No. 4,873,248.

EP-A 259 139 describes 2-(pyridin-2-yl)pyrimidines which have an optionally substituted phenyl group in the 6-position of the pyridine radical and which may have a fused saturated 5- or 6-membered ring in the 3,4-position of the pyrimidine ring. The compounds are likewise suitable for controlling phytopathogenic fungi (harmful fungi).

WO 2006/010570 describes fungicidally active 2-(6-phenylpyridin-2-yl)pyrimidine compounds of the formula B below:

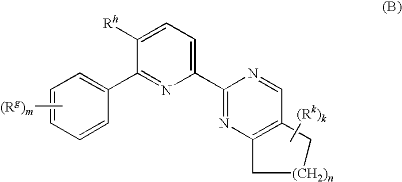

in which: k is 0, 1, 2 or 3, m is 0, 1, 2, 3, 4 or 5 and n is 1, 2, 3, 4 or 5, the substituents R^{g} are inter alia halogen, OH, CN, NO₂, C₁-C₄-alkyl, C₁-C₄-haloalkyl, C₁-C₄-alkoxy, C₁-C₄-haloalkoxy, C₂-C₄-alkenyl, C₂-C₄-alkynyl, C₃-C₈Cycloalkyl, C₁-C₄-alkoxy-C₁-C₄-alkyl, amino, phenoxy, etc., R^{h} is C₁-C₄-haloalkyl, C₁-C₄-alkoxy, C₁-C₄-haloalkoxy, hydroxyl, halogen, CN or NO₂ and R^{k} is C₁-C₄-alkyl.

With respect to their fungicidal activity, some of the 2-(pyridin-2-yl)pyrimidines known from the prior art are unsatisfactory, or they have unwanted properties such as low crop plant compatibility.

Accordingly, it is an object of the present invention to provide novel compounds having improved fungicidal activity and/or better compatibility with crop plants.

Surprisingly, this object is achieved by 3-(pyridin-2-yl)-[1,2,4]-triazine compounds of the general formula I

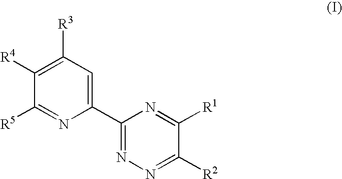

in which:
R¹, R² independently of one another are OH, halogen, NO₂, NH₂, C₁-C₈-alkyl, C₁-C₈-alkoxy, C₁-C₈-haloalkyl, C₁-C₈-haloalkoxy, C₁-C₈-alkylamino or di(C₁-C₈-alkyl)amino,
   or together with the carbon atoms to which they are attached may form a saturated 5-, 6- or 7-membered carbocycle or heterocycle which, in addition to the carbon ring members, has one or two heteroatoms selected from the group consisting of oxygen and sulfur as ring members, where the carbocycle and the heterocycle are unsubstituted or have 1, 2, 3 or 4 C₁-C₄-alkyl groups as substituents;
R³ is hydrogen, C₁-C₄-alkyl, C₁-C₄-alkoxy, C₁-C₄-haloalkyl, C₁-C₄-haloalkoxy, C₃-C₆-cycloalkyl, C₃-C₆-cycloalkylmethyl, or halogen;
R⁴ is hydrogen, C₁-C₄-alkyl, C₁-C₄-alkoxy, C₁-C₄-haloalkyl, C₁-C₄-haloalkoxy or halogen;
R⁵ is C₁-C₈-alkyl, C₁-C₈-haloalkyl, C₁-C₈-alkoxy, C₁-C₈-haloalkoxy, C₃-C₈-cycloalkyl, C₃-C₈-cycloalkyloxy, 5- or 6-membered heteroaryl, phenyl, phenoxy, benzyl, benzyloxy, 5- or 6-membered heteroarylmethyl or 5- or 6-membered heteroaryloxy, where the cyclic radicals mentioned above are unsubstituted or may have 1, 2, 3, 4 or 5 radicals R^{a}, where
R^{a} is selected from the group consisting of OH, SH, halogen, NO₂, NH₂, CN, COOH, C₁-C₈-alkyl, C₁-C₈-alkoxy, C₁-C₈-haloalkyl, C₁-C₈-haloalkoxy, C₁-C₈-alkylamino, di(C₁-C₈-alkyl)amino, C₁-C₈-alkylthio, C₁-C₈-haloalkylthio, C₁-C₈-alkylsulfinyl, C₁-C₈-haloalkylsulfinyl, C₁-C₈-alkylsulfonyl, C₁-C₈-haloalkylsulfonyl, C₃-C₈-cycloalkyl, phenyl, phenoxy and radicals of the formula C(=Z)R^{aa} in which Z is O, S, N(C₁-C₈-alkyl), N(C₁-C₈-alkoxy), N(C₃-C₈-alkenyloxy) or N(C₃-C₈-alkynyloxy) and R^{aa} is hydrogen, C₁-C₈-alkyl, C₁-C₈-alkoxy, NH₂, C₁-C₈-alkylamino or di(C₁-C₈-alkyl)amino, or two radicals R^{a} attached to adjacent carbon atoms together with the carbon atoms to which they are attached may also form a saturated 5-, 6- or 7-membered carbocycle, a benzene ring or a 5-, 6- or 7-membered heterocycle which, in addition to the carbon ring members, has one or two heteroatoms selected from the group consisting of oxygen and sulfur as ring members, where the carbocycle and the heterocycle are unsubstituted or have 1, 2, 3 or 4 C₁-C₄-alkyl groups as substituents;
   and the agriculturally useful salts of the compounds of the formula I.

Accordingly, the present invention provides the 3-(pyridin-2-yl)triazines of the general formula I and their agriculturally acceptable salts.

The present invention furthermore provides the use of the 3-(pyridin-2-yl)triazines of the general formula I and their agriculturally acceptable salts for controlling phytopathogenic fungi (=harmful fungi), and also a method for controlling phytopathogenic fungi wherein the fungi or the materials, plants, the soil or seed to be protected against fungal attack are/is treated with an effective amount of a compound of the general formula I and/or with an agriculturally acceptable salt of I.

The present invention furthermore provides a composition for controlling harmful fungi and comprising at least one 3-(pyridin-2-yl)triazine compound of the general formula I and/or an agriculturally acceptable salt thereof and at least one liquid or solid carrier.

Depending on the substitution pattern, the compounds of the formula I and their tautomers may have one or more centers of chirality, in which case they are present as pure enantiomers or pure diastereomers or as enantiomer or diastereomer mixtures. The invention provides both the pure enantiomers or diastereomers and their mixtures.

Agriculturally useful salts encompass especially the salts of those cations or the acid addition salts of those acids whose cations and anions, respectively, have no adverse effect on the fungicidal action of the compounds I. Suitable cations are thus in particular the ions of the alkali metals, preferably sodium and potassium, of the alkaline earth metals, preferably calcium, magnesium and barium, of the transition metals, preferably manganese, copper, zinc and iron, and also the ammonium ion which, if desired, may carry one to four C₁-C₄-alkyl substituents and/or one phenyl or benzyl substituent, preferably diisopropylammonium, tetramethylammonium, tetrabutylammonium, trimethylbenzylammonium, furthermore phosphonium ions, sulfonium ions, preferably tri(C₁-C₄-alkyl)sulfonium, and sulfoxonium ions, preferably tri(C₁-C₄-alkyl)sulfoxonium.

Anions of useful acid addition salts are primarily chloride, bromide, fluoride, hydrogensulfate, sulfate, dihydrogenphosphate, hydrogenphosphate, phosphate, nitrate, bicarbonate, carbonate, hexafluorosilicate, hexafluorophosphate, benzoate, and the anions of C₁-C₄-alkanoic acids, preferably formate, acetate, propionate and butyrate. They can be formed by reacting I with an acid of the corresponding anion, preferably of hydrochloric acid, hydrobromic acid, sulfuric acid, phosphoric acid or nitric acid.

In the definitions of the variables given in the formulae above, collective terms are used which are generally representative for the substituents in question. The term Cₙ-Cₘ indicates the number of carbon atoms possible in each case in the substituent or substituent moiety in question:

halogen: fluorine, chlorine, bromine and iodine;

alkyl and also all alkyl moieties in alkoxy, alkoxyalkyl, alkylcarbonyl, alkoxycarbonyl, alkylthio, alkylsulfonyl, alkylsulfinyl, alkylamino, dialkylamino, alkylaminocarbonyl, dialkylaminocarbonyl: saturated, straight-chain or branched hydrocarbon radicals having 1 to 8 (C₁-C₈-alkyl), frequently 1 to 6 (C₁-C₆-alkyl) and in particular 1 to 4 carbon atoms (C₁-C₄-alkyl), such as methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl, heptyl, 1-methylhexyl, octyl, 1-methylheptyl and 2-ethylhexyl;

haloalkyl and also all haloalkyl moieties in haloalkoxy and haloalkylthio: straight-chain or branched alkyl groups having 1 to 8 and in particular 1 to 4 carbon atoms (as mentioned above), where some or all of the hydrogen atoms in these groups may be replaced by halogen atoms as mentioned above, in particular fluorine or chlorine: in particular C₁-C₂-haloalkyl such as chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl and 1,1,1-trifluoroprop-2-yl;

alkenyl: monounsaturated, straight-chain or branched hydrocarbon radicals having 2 to 8 or 3 to 8 carbon atoms and a double bond in any position, for example ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl;

alkynyl: straight-chain or branched hydrocarbon groups having 2 to 8 or 3 to 8 carbon atoms and a triple bond in any position, for example ethynyl, 1-propynyl, 2-propynyl, 1-butynyl, 2-butynyl, 3-butynyl, 1-methyl-2-propynyl;

cycloalkyl: monocyclic saturated hydrocarbon groups having 3 to 8, preferably to 6, carbon ring members, such as cyclopropyl, cyclobutyl, cyclopentyl and cyclohexyl;

cycloalkylmethyl: a cycloalkyl radical as mentioned above which is attached via a methylene group (CH₂);

alkylamino and also the alkylamino moieties in alkylaminocarbonyl: an alkyl group which is attached via an NH group, where alkyl is one of the alkyl radicals mentioned above having 1 to 8 carbon atoms, such as methylamino, ethylamino, n-propylamino, isopropylamino, n-butylamino and the like;

dialkylamino and also the dialkylamino moieties in dialkylaminocarbonyl: a radical of the formula N(alkyl)₂, where alkyl is one of the alkyl radicals mentioned above having 1 to 8 carbon atoms, for example dimethylamino, diethylamino, methylethylamino, N-methyl-N-propylamino and the like;

alkoxy and also the alkoxy moieties in alkoxycarbonyl: an alkyl group, attached via an oxygen, having 1 to 8, in particular 1 to 6 and especially 1 to 4 carbon atoms, for example methoxy, ethoxy, n-propoxy, 1-methylethoxy, butoxy, 1-methylpropoxy, 2-methylpropoxy or 1,1-dimethylethoxy;

alkoxycarbonyl: an alkoxy radical as mentioned above, attached via a carbonyl group;

alkylthio: an alkyl group as mentioned above, attached via a sulfur atom;

alkylsulfinyl: an alkyl group as mentioned above, attached via an S(═O) group;

alkylsulfonyl: an alkyl group as mentioned above, attached via an S(═O)₂ group;

haloalkoxy: an alkoxy radical having 1 to 8, in particular 1 to 6 and especially 1 to 4 carbon atoms as mentioned above which is partially or fully substituted by fluorine, chlorine, bromine and/or iodine, preferably substituted by fluorine, i.e., for example, OCH₂F, OCHF₂, OCF₃, OCH₂Cl, OCHCl₂, OCCl₃, chlorofluoromethoxy, dichlorofluoromethoxy, chlorodifluoromethoxy, 2-fluoroethoxy, 2-chloroethoxy, 2-bromoethoxy, 2-iodoethoxy, 2,2-difluoroethoxy, 2,2,2-trifluoroethoxy, 2-chloro-2-fluoroethoxy, 2-chloro-2,2-difluoroethoxy, 2,2-dichloro-2-fluoroethoxy, 2,2,2-trichloroethoxy, OC₂F₅, 2-fluoropropoxy, 3-fluoropropoxy, 2,2-difluoropropoxy, 2,3-difluoropropoxy, 2-chloropropoxy, 3-chloropropoxy, 2,3-dichloropropoxy, 2-bromopropoxy, 3-bromopropoxy, 3,3,3-trifluoropropoxy, 3,3,3-trichloropropoxy, OCH₂—C₂F₅, OCF₂—C₂F₅, 1-(CH₂F)-2-fluoroethoxy, 1-(CH₂Cl)-2-chloroethoxy, 1-(CH₂Br)-2-bromoethoxy, 4-fluorobutoxy, 4-chlorobutoxy, 4-bromobutoxy or nonafluorobutoxy;

alkylene: a straight-chain saturated hydrocarbon chain having 2 to 6 and in particular 2 to 4 carbon atoms, such as ethane-1,2-diyl, propane-1,3-diyl, butane-1,4-diyl, pentane-1,5-diyl or hexane-1,6-diyl.

Saturated 5-, 6- or 7-membered heterocycle which has one or two heteroatoms selected from the group consisting of oxygen and sulfur as ring members: a ring constructed of carbon atoms and 1 or 2 heteroatoms selected from the group consisting of oxygen and sulfur, the total number of ring atoms (ring members) being 5, 6 or 7, for example: oxolane, oxepane, oxane (tetrahydropyran), 1,3-dioxolane, 1,3-dioxane, 1,4-dioxane, thiolane, thiane, thiepane, 1,3-dithiolane, 1,3-dithiane and 1,4-dithiane;

5- or 6-membered heteroaryl: a 5- or 6-membered aromatic ring which, in addition to carbon, has 1, 2, 3 or 4 heteroatoms as ring members, the heteroatoms typically being selected from the group consisting of oxygen, nitrogen and sulfur, in particular:
5-membered heteroaryl which has 1, 2, 3 or 4 nitrogen atoms as ring members, such as 1-, 2- or 3-pyrrolyl, 1-, 3- or 4-pyrazolyl, 1-, 2- or 4-imidazolyl, 1,2,3-[1H]-triazol-1-yl, 1,2,3-[2H]-triazol-2-yl, 1,2,3-[1H]-triazol-4-yl, 1,2,3-[1H]-triazol-5-yl, 1,2,3-[2H]-triazol-4-yl, 1,2,4-[1H]-triazol-1-yl, 1,2,4-[1H]-triazol-3-yl, 1,2,4-[1H]-triazol-5-yl, 1,2,4-[4H]-triazol-4-yl, 1,2,4-[4H]-triazol-3-yl, [1H]-tetrazol-1-yl, [1H]-tetrazol-5-yl, [2H]-tetrazol-2-yl and [2H]-tetrazol-5-yl;
5-membered heteroaryl which has 1 heteroatom selected from the group consisting of oxygen and sulfur and optionally 1, 2 or 3 nitrogen atoms as ring members, for example 2-furyl, 3-furyl, 2-thienyl, 3-thienyl, 3- or 4-isoxazolyl, 3- or 4-isothiazolyl, 2-, 4- or 5-oxazolyl, 2-, 4 or 5-thiazolyl, 1,2,4-thiadiazol-3-yl, 1,2,4-thiadiazol-5-yl, 1,3,4-thiadiazol-2-yl, 1,2,4-oxadiazol-3-yl, 1,2,4-oxadiazol-5-yl and 1,3,4-oxadiazol-2-yl;
6-membered heteroaryl which has 1, 2, 3 or 4 nitrogen atoms as ring members, such as 2-pyridinyl, 3-pyridinyl, 4-pyridinyl, 2-pyrimidinyl, 4-pyrimidinyl, 5-pyrimidinyl, 2-pyrazinyl, 3-pyridazinyl, 4-pyridazinyl, 1,2,4-triazin-3-yl, 1,2,4-triazin-5-yl, 1,2,4-triazin-6-yl and 1,3,5-triazinyl.

With a view to the use as fungicides, preference is given to those compounds of the formula I in which the variables R¹, R², R³, R⁴ and R⁵ independently of one another and in particular in combination have the following meanings:

According to a first embodiment of the present invention, R¹ and R² independently of one another are monovalent radicals. Here, R¹ and R² may be identical of different. In this case, they are preferably selected from the group consisting of fluorine, chlorine, C₁-C₄-alkyl, especially methyl, ethyl or n-propyl, furthermore methoxy, ethoxy, CF₃, CHF₂, OCF₃ and OCHF₂.

According to a second embodiment of the present invention, R¹ and R² together with the carbon atoms to which they are attached form a saturated 5-, 6- or 7-membered carbocycle or heterocycle which is as defined above and which may carry one or more C₁-C₄-alkyl groups as substituents. In this embodiment, R¹ and R² together with the carbon atoms of the triazine ring to which they are attached are preferably one of the following rings:

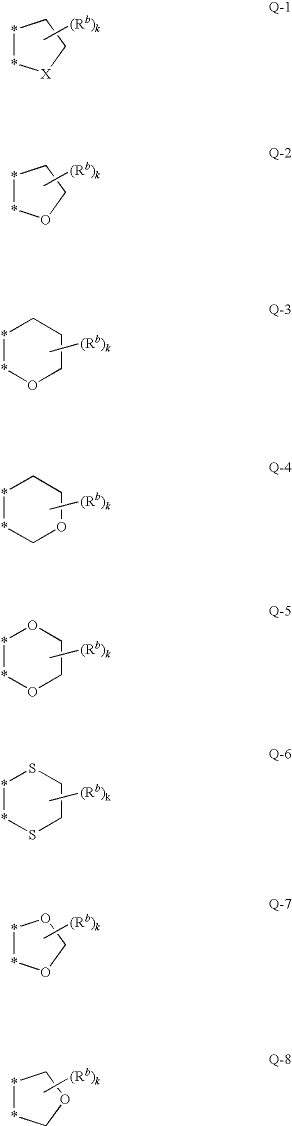

in which
* are the atoms of the triazine ring;
k is 0, 1, 2, 3 or 4;
R^{b} is C₁-C₄-alkyl, in particular methyl; and
X is (CH₂)ₙ where n=1, 2 or 3.

The radicals R^{b} can be located at any carbon atoms of these rings, and, if k≠0, for example, 1, 2, 3 or 4 of the hydrogen atoms in (CH₂)ₙ may be replaced by R^{b}. The radicals Q-2, Q-3 and Q-4 can assume any orientation with respect to the triazine ring. From among the radicals Q-1 to Q-8, particular preference is given to the radical Q-1 and especially to radicals Q-1 where n=2 or 3. The variable k is in particular 0, 1 or 2.

R³ is preferably hydrogen, fluorine, chlorine, C₁-C₄-alkyl, especially methyl, ethyl, isopropyl or tert-butyl, methoxy, ethoxy, CF₃, CHF₂, OCF₃ or OCHF₂, in particular hydrogen or methyl. Particular preference is furthermore given to compounds of the formula I in which R³ is chlorine. Particular preference is furthermore given to compounds of the formula I in which R³ is CF₃. Particular preference is furthermore given to compounds of the formula I in which R³ is methoxy or ethoxy.

R⁴ is preferably hydrogen, fluorine, chlorine, C₁-C₄-alkyl, especially methyl or ethyl, methoxy, ethoxy, CF₃, CHF₂, OCF₃ or OCHF₂. R⁴ is in particular hydrogen, fluorine, chlorine or methyl.

In preferred compounds of the formula I, R⁵ is phenyl, phenoxy or benzyl, where the phenyl ring in the three radicals mentioned above is unsubstituted or has 1, 2, 3, 4 or 5 radicals R^{a}, in particular 1, 2 or 3 radicals R^{a}.

Preferred radicals R^{a} are selected from the group consisting of halogen, C₁-C₄-alkyl, C₁-C₂-haloalkyl, C₁-C₄-alkoxy, C₁-C₂-haloalkoxy, C₁-C₄-alkylthio, C₁-C₄-alkylcarbonyl, C₁-C₄-alkoxycarbonyl, and radicals of the formula C(═N—O—C₁-C₈-alkyl)R^{aa} in which R^{aa} is hydrogen or C₁-C₄-alkyl. Especially preferably, the radicals R^{a} are selected from the group consisting of halogen, especially chlorine or fluorine, methyl, methoxy, trifluoromethyl, difluoromethyl, trifluoromethoxy, difluoromethoxy and methylthio.

According to a preferred embodiment of the invention, R⁵ in formula I is phenyl, phenoxy or benzyl, where the phenyl ring has 1, 2, 3, 4 or 5 and in particular 1, 2 or 3 radicals R^{a}, where the radicals R^{a} are preferably selected from the radicals R^{a} mentioned as being preferred and in particular from the radicals R^{a} mentioned as being particularly preferred. In this embodiment, the phenyl radical in phenyl, phenoxy or benzyl is a radical of the formula P:

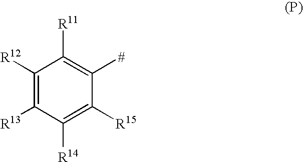

in which # is the point of attachment to the pyridine ring and R¹¹, R¹², R¹³, R¹⁴ and R¹⁵ are hydrogen or at least one of these radicals, for example 1, 2, 3, 4 or 5 of these radicals, has/have one of the meanings given for R^{a}, in particular one of the meanings given as being preferred or particularly preferred. In a preferred embodiment, at least one and especially 1, 2 or 3 of the radicals R¹¹, R¹², R¹³, R¹⁴ or R¹⁵ is/are different from hydrogen. In particular:
R¹¹ is hydrogen, fluorine, chlorine, CH₃, OCH₃, OCHF₂, OCF₃ or CF₃;
R¹², R¹⁴ independently of one another are hydrogen, chlorine, fluorine, CH₃, OCH₃, OCHF₂, OCF₃ or CF₃, where one of the radicals R¹² and R¹⁴ may also be NO₂, C(O)CH₃ or COOCH₃; in particular, R¹² and R¹⁴ are hydrogen, fluorine, methyl or trifluoromethyl;
R¹³ is hydrogen, fluorine, chlorine, cyano, OH, CHO, NO₂, NH₂, methylamino, dimethylamino, diethylamino, C₁-C₄-alkyl, especially CH₃, C₂H₅, CH(CH₃)₂, C₃-C₈-cycloalkyl, especially cyclopropyl, cyclopentyl or cyclohexyl, C₁-C₄-alkoxy, especially OCH₃, C₁-C₄-alkylthio, especially methylthio or ethylthio, C₁-C₄-haloalkyl, especially CF₃, C₁-C₄-haloalkoxy, especially OCHF₂ or OCF₃, or CO(A²) where A² is C₁-C₄-alkyl, especially methyl, or C₁-C₄-alkoxy, especially OCH₃, or a group C(R^{13a})═NOR^{13b} in which R^{13a} is hydrogen or methyl and R^{13b} is C₁-C₄-alkyl, propargyl or allyl or R¹² and R¹³ together form a group O—CH₂—O; and
R¹⁵ is hydrogen, fluorine, chlorine, or C₁-C₄-alkyl, especially CH₃, in particular hydrogen or fluorine.

Advantageously, if more than one of the radicals R¹¹, R¹², R¹³, R¹⁴ or R¹⁵ is different from hydrogen, then only one of the radicals different from hydrogen is different from halogen or methyl. Especially if one of the radicals R¹¹, R¹², R¹³, R¹⁴ or R¹⁵ is different from hydrogen, halogen or methyl, the remaining radicals R¹¹, R¹², R¹³, R¹⁴, R¹⁵ are selected from the group consisting of halogen and hydrogen.

Examples of radicals P are the radicals mentioned below: phenyl, 2-fluorophenyl, 3-fluorophenyl, 4-fluorophenyl, 2-chlorophenyl, 3-chlorophenyl, 4-chlorophenyl, 3-bromophenyl, 4-bromophenyl, 2-trifluoromethylphenyl, 3-trifluoromethylphenyl, 4-trifluoromethylphenyl, 2-(methylthio)phenyl, 3-(methylthio)phenyl, 4-(methylthio)phenyl, 2-methoxyphenyl, 3-methoxyphenyl, 4-methoxyphenyl, 4-nitrophenyl, 4-cyanophenyl, 4-tert-butylphenyl, 4-isopropylphenyl, 3-ethoxyphenyl, 4-ethoxyphenyl, 4-n-propoxyphenyl, 4-isopropoxyphenyl, 3-isopropoxyphenyl, 4-n-butoxyphenyl, 4-tert-butoxyphenyl, 4-acetylphenyl, 4-methoxycarbonylphenyl, 4-ethoxycarbonylphenyl, 4-tert-butoxycarbonylphenyl, 4-(methoxyiminomethyl)phenyl, 4-(1-(methoxyimino)ethyl)phenyl, 2,3-difluorophenyl, 2,4-difluorophenyl, 2,5-difluorophenyl, 3,4-difluorophenyl, 3,5-difluorophenyl, 2,6-difluorophenyl, 2,4,6-trifluorophenyl, 2,4,5-trifluorophenyl, 2,3,4-trifluorophenyl, 2,3,5-trifluorophenyl, 3,4,5-trifluorophenyl, 2,3-dichlorophenyl, 2,5-dichlorophenyl, 3,5-dichlorophenyl, 2,6-dichlorophenyl, 2,3-dimethylphenyl, 2,4-dimethylphenyl, 2,5-dimethylphenyl, 2,4,5-trimethylphenyl, 2,3-dimethoxyphenyl, 2,4-dimethoxyphenyl, 3,4-dimethoxyphenyl, 2,4-bis(trifluoromethyl)phenyl, 3,5-bis(trifluoromethyl)phenyl, 2-methyl-3-methoxyphenyl, 2-methyl-4-methoxyphenyl, 2-methyl-6-methoxyphenyl, 3-chloro-4-fluorophenyl, 2-chloro-4-fluorophenyl, 2-chloro-6-fluorophenyl, 4-chloro-2-fluorophenyl, 5-chloro-2-fluorophenyl, 4-fluoro-3-methylphenyl, 2-fluoro-4-methylphenyl, 4-fluoro-2-methylphenyl, 2-fluoro-3-methoxyphenyl, 2-fluoro-4-methoxyphenyl, 2-fluoro-6-methoxyphenyl, 2-fluoro-4-trifluoromethylphenyl, 4-chloro-3-methylphenyl, 2-chloro-4-methylphenyl, 2-chloro-6-methylphenyl, 3-chloro-2-methylphenyl, 5-chloro-2-methylphenyl, 2-chloro-4-methoxyphenyl, 2-chloro-6-methoxyphenyl, 2-chloro-4-trifluoromethylphenyl, 3-fluoro-4-methylphenyl, 4-fluoro-3-methylphenyl, 3-fluoro-4-methoxyphenyl, 3-fluoro-4-ethoxyphenyl, 3-fluoro-4-trifluoromethylphenyl, 3-chloro-4-methylphenyl, 3-chloro-4-methoxyphenyl, 3-chloro-4-ethoxyphenyl, 3-chloro-4-trifluoromethylphenyl, 3-methyl-4-methoxyphenyl, 4-chloro-2,5-difluorophenyl, 4-tert-butyl-2-fluorophenyl, 2-fluoro-4-isopropylphenyl, 4-ethoxy-2-fluorophenyl, 4-acetyl-2-fluorophenyl, 2-methylphenyl, 3-methylphenyl, 4-methylphenyl, 2-ethylphenyl, 3-ethylphenyl, 4-ethylphenyl.

Particularly preferably, R⁵ in formula I is phenyl and especially a radical P and in particular one of the radicals P shown here as an example.

According to a further preferred embodiment, R⁵ is C₁-C₆-alkyl or C₁-C₆-haloalkyl, in particular C₃-C₆-alkyl, especially n-propyl, isopropyl, tert-butyl, 1,2-dimethylpropyl or 1,2,2-trimethylpropyl, or trifluoromethyl.

According to a further preferred embodiment, R⁵ is 5-membered heteroaryl which, in addition to carbon, has 1, 2, 3 or 4 nitrogen atoms as ring atoms; or 5-membered heteroaryl which, in addition to carbon, has 1 heteroatom selected from the group consisting of oxygen and sulfur and optionally 1, 2 or 3 nitrogen atoms as ring atoms, or is 6-membered hetaryl which has 1, 2, 3 or 4 nitrogen atoms as ring atoms, where the 5- and 6-membered hetaryl may be unsubstituted or some or all of the hydrogen atoms in the unsubstituted hetaryl may be replaced by substituents R^{a} of the type indicated above, so that the total number of all substituents R^{a} on hetaryl is typically 1, 2, 3 or 4. Substituents on nitrogen ring atoms are in particular radicals R^{a} attached via carbon and especially C₁-C₄-alkyl.

In this embodiment, R⁵ is preferably optionally substituted 2-furyl, 3-furyl, 2-thienyl, 3-thienyl, 2-pyridyl, 3-pyridyl, 4-pyridyl, 2-pyrimidinyl, 4-pyrimidinyl or 5-pyrimidinyl, where the heterocyclic radicals mentioned above are preferably unsubstituted or have 1, 2 or 3 substituents R^{a}. With respect to the preferred and particularly preferred radicals, what has been said above applies.

Examples of preferred heteroaromatic radicals R⁵ are
optionally substituted 2-thienyl, such as unsubstituted 2-thienyl, 5-methylthiophen-2-yl, 4-methylthiophen-2-yl, 5-chlorothiophen-2-yl, 3-cyanothiophen-2-yl, 5-formylthiophen-2-yl, 5-acetylthiophen-2-yl, 5-(methoxyiminomethyl)thiophen-2-yl, 5-(1-(methoxyimino)ethyl)thiophen-2-yl, 4-bromothiophen-2-yl, 3,5-dichlorothiophen-2-yl,
optionally substituted 3-thienyl, such as unsubstituted 3-thienyl, 2-methylthiophen-3-yl, 2,5-dichlorothiophen-3-yl, 2,4,5-trichlorothiophen-3-yl,
optionally substituted 2-furyl, such as unsubstituted 2-furyl, 5-methylfuran-2-yl, 5-chlorofuran-2-yl, 4-methylfuran-2-yl, 3-cyanofuran-2-yl, 5-acetylfuran-2-yl,
optionally substituted 3-furyl, such as unsubstituted 3-furyl, 2-methylfuran-3-yl, 2,5-dimethylfuran-3-yl,
optionally substituted 2-pyridyl, such as unsubstituted 2-pyridyl, 3-fluoropyridin-2-yl, 3-chloropyridin-2-yl, 3-bromopyridin-2-yl, 3-trifluoromethyl-pyridin-2-yl, 3-methylpyridin-2-yl, 3-ethylpyridin-2-yl, 3,5-difluoropyridin-2-yl, 3,5-dichloropyridin-2-yl, 3,5-dibromopyridin-2-yl, 3,5-dimethylpyridin-2-yl, 3-fluoro-5-trifluoromethylpyridin-2-yl, 3-chloro-5-fluoropyridin-2-yl, 3-chloro-5-methylpyridin-2-yl, 3-fluoro-5-chloropyridin-2-yl, 3-fluoro-5-methylpyridin-2-yl, 3-methyl-5-fluoropyridin-2-yl, 3-methyl-5-chloropyridin-2-yl, 5-nitropyridin-2-yl, 5-cyanopyridin-2-yl, 5-methoxycarbonylpyridin-2-yl, 5-trifluoromethylpyridin-2-yl, 5-methylpyridin-2-yl, 4-methylpyridin-2-yl, 6-methylpyridin-2-yl,
optionally substituted 3-pyridyl, such as unsubstituted 3-pyridyl, 2-chloropyridin-3-yl, 2-bromopyridin-3-yl, 2-methylpyridin-3-yl, 2,4-dichloropyridin-3-yl, 2,4-dibromopyridin-3-yl, 2,4-difluoropyridin-3-yl, 2-fluoro-4-chloropyridin-3-yl, 2-chloro-4-fluoropyrdin-3-yl, 2-chloro-4-methylpyridin-3-yl, 2-methyl-4-fluoropyridin-3-yl, 2-methyl-4-chloropyridin-3-yl, 2,4-dimethylpyridin-3-yl, 2,4,6-trichloropyridin-3-yl, 2,4,6-tribromopyridin-3-yl, 2,4,6-trimethylpyridin-3-yl, 2,4-dichloro-6-methylpyridin-3-yl, 6-methoxypyridin-3-yl, 6-chloropyridin-3-yl,
optionally substituted 4-pyridyl, such as unsubstituted 4-pyridyl, 3-chloropyridin-4-yl, 3-bromopyridin-4-yl, 3-methylpyridin-4-yl, 3,5-dichloropyridin-4-yl, 3,5-dibromopyridin-4-yl, 3,5-dimethylpyridin-4-yl,
optionally substituted 4-pyrimidinyl, such as unsubstituted 4-pyrimidinyl, 5-chloropyrimidin-4-yl, 5-fluoropyrimidin-4-yl, 5-fluoro-6-chloropyrimidin-4-yl, 2-methyl-6-trifluoromethylpyrimidin-4-yl, 2,5-dimethyl-6-trifluoromethylpyrimidin-4-yl, 5-methyl-6-trifluoromethyl-pyrimidin-4-yl, 6-trifluoromethylpyrimidin-4-yl, 2-methyl-5-fluoropyrimidin-4-yl, 2-methyl-5-chloropyrimidin-4-yl, 5-chloro-6-methylpyrimidin-4-yl, 5-chloro-6-ethylpyrimidin-4-yl, 5-chloro-6-isopropylpyrimidin-4-yl, 5-bromo-6-methylpyrimidin-4-yl, 5-fluoro-6-methylpyrimidin-4-yl, 5-fluoro-6-fluoromethylpyrimidin-4-yl, 2,6-dimethyl-5-chloropyrimdin-4-yl, 5,6-dimethylpyrimidin-4-yl, 2,5-dimethylpyrimidin-4-yl, 2,5,6-trimethylpyrimidin-4-yl, 5-methyl-6-methoxypyrimidin-4-yl,
optionally substituted 5-pyrimidinyl, such as unsubstituted 5-pyrimidinyl, 4-methylpyrimidin-5-yl, 4,6-dimethylpyrimidin-5-yl, 2,4,6-trimethylpyrimidin-5-yl, 4-trifluoromethyl-6-methylpyrimidin-5-yl,
optionally substituted 2-pyrimidinyl, such as unsubstituted 2-pyrimidinyl, 4,6-dimethylpyrimidin-2-yl, 4,5,6-trimethylpyrimidin-2-yl, 4,6-ditrifluoromethylpyrimidin-2-yl and 4,6-dimethyl-5-chloropyrimidin-2-yl.

Especially preferred are the following groups of compounds of the formula I:

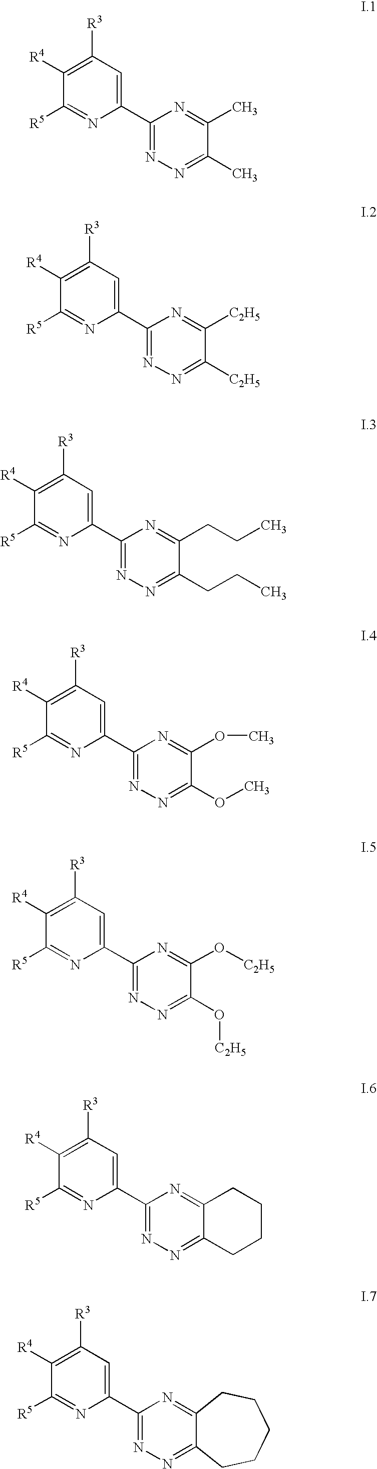

In particular with a view to their use, preference is given to the compounds of the general formulae I.1, I.2, I.3, I.4, I.5, I.6 and I.7 mentioned in Tables 1 to 9.

Table 1

Compounds of the formulae I.1, I.3, I.4, I.5, I.6 and I.7 in which R³ is hydrogen and the combination of R⁴ and R⁵ for a compound corresponds in each case to one row of Table A.

Table 2

Compounds of the formulae I.1, I.3, I.4, I.5, I.6 and I.7 in which R³ is methyl and the combination of R⁴ and R⁵ for a compound corresponds in each case to one row of Table A.

Table 3

Compounds of the formulae I.1, I.3, I.4, I.5, I.6 and I.7 in which R³ is ethyl and the combination of R⁴ and R⁵ for a compound corresponds in each case to one row of Table A.

Table 4

Compounds of the formulae I.1, I.3, I.4, I.5, I.6 and I.7 in which R³ is methoxy and the combination of R⁴ and R⁵ for a compound corresponds in each case to one row of Table A.

Table 5

Compounds of the formulae I.1, I.3, I.4, I.5, I.6 and I.7 in which R³ is ethoxy and the combination of R⁴ and R⁵ for a compound corresponds in each case to one row of Table A.

Table 6

Compounds of the formulae I.1, I.3, I.4, I.5, I.6 and I.7 in which R³ is isopropyl and the combination of R⁴ and R⁵ for a compound corresponds in each case to one row of Table A.

Table 7

Compounds of the formulae I.1, I.3, I.4, I.5, I.6 and I.7 in which R³ is tert-butyl and the combination of R⁴ and R⁵ for a compound corresponds in each case to one row of Table A.

Table 8

Compounds of the formulae I.1, I.3, I.4, I.5, I.6 and I.7 in which R³ is trifluoromethyl and the combination of R⁴ and R⁵ for a compound corresponds in each case to one row of Table A.

Table 9

Compounds of the formulae I.1, I.3, I.4, I.5, I.6 and I.7 in which R³ is difluoromethoxy and the combination of R⁴ and R⁵ for a compound corresponds in each case to one row of Table A.

TABLE A

| No. | R⁴ | R⁵ |
|---|---|---|
| A-1. | H | phenyl |
| A-2. | H | 2-fluorophenyl |
| A-3. | H | 3-fluorophenyl |
| A-4. | H | 4-fluorophenyl |
| A-5. | H | 2-chlorophenyl |
| A-6. | H | 3-chlorophenyl |
| A-7. | H | 4-chlorophenyl |
| A-8. | H | 3-bromophenyl |
| A-9. | H | 4-bromophenyl |
| A-10. | H | 2-trifluoromethylphenyl |
| A-11. | H | 3-trifluoromethylphenyl |
| A-12. | H | 4-trifluoromethylphenyl |
| A-13. | H | 2-(methylthio)phenyl |
| A-14. | H | 3-(methylthio)phenyl |
| A-15. | H | 4-(methylthio)phenyl |
| A-16. | H | 2-methoxyphenyl |
| A-17. | H | 3-methoxyphenyl |
| A-18. | H | 4-methoxyphenyl |
| A-19. | H | 4-nitrophenyl |
| A-20. | H | 4-cyanophenyl |
| A-21. | H | 4-tert-butylphenyl |
| A-22. | H | 4-isopropylphenyl |
| A-23. | H | 3-ethoxyphenyl |
| A-24. | H | 4-ethoxyphenyl |
| A-25. | H | 4-n-propoxyphenyl |
| A-26. | H | 3-isopropoxyphenyl |
| A-27. | H | 4-isopropoxyphenyl |
| A-28. | H | 4-n-butoxyphenyl |
| A-29. | H | 4-tert-butoxyphenyl |
| A-30. | H | 4-(methoxyiminomethyl)phenyl |
| A-31. | H | 4-(1-(methoxyimino)ethyl)phenyl |
| A-32. | H | 2,3-difluorophenyl |
| A-33. | H | 2,4-difluorophenyl |
| A-34. | H | 2,5-difluorophenyl |
| A-35. | H | 3,4-difluorophenyl |
| A-36. | H | 3,5-difluorophenyl |
| A-37. | H | 2,6-difluorophenyl |
| A-38. | H | 2,4,6-trifluorophenyl |
| A-39. | H | 2,4,5-trifluorophenyl |
| A-40. | H | 2,3,4-trifluorophenyl |
| A-41. | H | 2,3,5-trifluorophenyl |
| A-42. | H | 3,4,5-trifluorophenyl |
| A-43. | H | 2,3-dichlorophenyl |
| A-44. | H | 2,5-dichlorophenyl |
| A-45. | H | 3,5-dichlorophenyl |
| A-46. | H | 2,6-dichlorophenyl |
| A-47. | H | 2,3-dimethylphenyl |
| A-48. | H | 2,4-dimethylphenyl |
| A-49. | H | 2,5-dimethylphenyl |
| A-50. | H | 2,4,5-trimethylphenyl |
| A-51. | H | 2,3-dimethoxyphenyl |
| A-52. | H | 2,4-dimethoxyphenyl |
| A-53. | H | 3,4-dimethoxyphenyl |
| A-54. | H | 2,4-bis(trifluoromethyl)phenyl |
| A-55. | H | 3,5-bis(trifluoromethyl)phenyl |
| A-56. | H | 2-methyl-3-methoxyphenyl |
| A-57. | H | 2-methyl-4-methoxyphenyl |
| A-58. | H | 2-methyl-6-methoxyphenyl |
| A-59. | H | 3-chloro-4-fluorophenyl |
| A-60. | H | 2-chloro-4-fluorophenyl |
| A-61. | H | 2-chloro-6-fluorophenyl |
| A-62. | H | 4-chloro-2-fluorophenyl |
| A-63. | H | 5-chloro-2-fluorophenyl |
| A-64. | H | 4-fluoro-3-methylphenyl |
| A-65. | H | 2-fluoro-4-methylphenyl |
| A-66. | H | 4-fluoro-2-methylphenyl |
| A-67. | H | 2-fluoro-3-methoxyphenyl |
| A-68. | H | 2-fluoro-4-methoxyphenyl |
| A-69. | H | 2-fluoro-6-methoxyphenyl |
| A-70. | H | 2-fluoro-4-trifluoromethylphenyl |
| A-71. | H | 4-chloro-3-methylphenyl |
| A-72. | H | 2-chloro-4-methylphenyl |
| A-73. | H | 2-chloro-6-methylphenyl |
| A-74. | H | 5-chloro-2-methylphenyl |
| A-75. | H | 3-chloro-2-methylphenyl |
| A-76. | H | 2-chloro-4-methoxyphenyl |
| A-77. | H | 2-chloro-6-methoxyphenyl |
| A-78. | H | 2-chloro-4-trifluoromethylphenyl |
| A-79. | H | 3-fluoro-4-methylphenyl |
| A-80. | H | 3-fluoro-4-isopropylphenyl |
| A-81. | H | 4-fluoro-3-methylphenyl |
| A-82. | H | 3-fluoro-4-methoxyphenyl |
| A-83. | H | 3-fluoro-4-ethoxyphenyl |
| A-84. | H | 3-fluoro-4-trifluoromethylphenyl |
| A-85. | H | 3-chloro-4-methylphenyl |
| A-86. | H | 3-chloro-4-methoxyphenyl |
| A-87. | H | 3-chloro-4-ethoxyphenyl |
| A-88. | H | 3-chloro-4-trifluoromethylphenyl |
| A-89. | H | 3-methyl-4-methoxyphenyl |
| A-90. | H | 4-chloro-2,5-difluorophenyl |
| A-91. | H | 4-tert-butyl-2-fluorophenyl |
| A-92. | H | 2-fluoro-4-isopropylphenyl |
| A-93. | H | 4-ethoxy-2-fluorophenyl |
| A-94. | H | 4-acetyl-2-fluorophenyl |
| A-95. | H | 2-thienyl |
| A-96. | H | 5-methylthiophen-2-yl |
| A-97. | H | 4-methylthiophen-2-yl |
| A-98. | H | 5-chlorothiophen-2-yl |
| A-99. | H | 3-cyanothiophen-2-yl |
| A-100. | H | 5-formylthiophen-2-yl |
| A-101. | H | 5-acetylthiophen-2-yl |
| A-102. | H | 5-(methoxyiminomethyl)thiophen-2-yl |
| A-103. | H | 5-(1-(methoxyimino)ethyl)thiophen-2-yl |
| A-104. | H | 4-bromothiophen-2-yl |
| A-105. | H | 3,5-dichlorothiophen-2-yl |
| A-106. | H | 3-thienyl |
| A-107. | H | 2-methylthiophen-3-yl |
| A-108. | H | 2,5-dichlorothiophen-3-yl |
| A-109. | H | 2,4,5-trichloro-thiophen-3-yl |
| A-110. | H | 2-furyl |
| A-111. | H | 5-methylfuran-2-yl |
| A-112. | H | 5-chlorofuran-2-yl |
| A-113. | H | 4-methylfuran-2-yl |
| A-114. | H | 3-cyanofuran-2-yl |
| A-115. | H | 5-acetylfuran-2-yl |
| A-116. | H | 3-furyl |
| A-117. | H | 2-methylfuran-3-yl |
| A-118. | H | 2,5-dimethylfuran-3-yl |
| A-119. | H | 2-pyridyl |
| A-120. | H | 3-fluoropyridin-2-yl |
| A-121. | H | 3-chloropyridin-2-yl |
| A-122. | H | 3-bromopyridin-2-yl |
| A-123. | H | 3-trifluoromethylpyridin-2-yl |
| A-124. | H | 3-methylpyridin-2-yl |
| A-125. | H | 3-ethylpyridin-2-yl |
| A-126. | H | 3,5-difluoropyridin-2-yl |
| A-127. | H | 3,5-dichloropyridin-2-yl |
| A-128. | H | 3,5-dibromopyridin-2-yl |
| A-129. | H | 3,5-dimethylpyridin-2-yl |
| A-130. | H | 3-fluoro-5-trifluoromethylpyridin-2-yl |
| A-131. | H | 3-chloro-5-fluoropyridin-2-yl |
| A-132. | H | 3-chloro-5-methylpyridin-2-yl |
| A-133. | H | 3-fluoro-5-chloropyridin-2-yl |
| A-134. | H | 3-fluoro-5-methylpyridin-2-yl |
| A-135. | H | 3-methyl-5-fluoropyridin-2-yl |
| A-136. | H | 3-methyl-5-chloropyridin-2-yl |
| A-137. | H | 5-nitropyridin-2-yl |
| A-138. | H | 5-cyanopyridin-2-yl |
| A-139. | H | 5-methoxycarbonylpyridin-2-yl |
| A-140. | H | 5-trifluoromethylpyridin-2-yl |
| A-141. | H | 5-methylpyridin-2-yl |
| A-142. | H | 4-methylpyridin-2-yl |
| A-143. | H | 6-methylpyridin-2-yl |
| A-144. | H | 3-pyridyl |
| A-145. | H | 2-chloropyridin-3-yl |
| A-146. | H | 2-bromopyridin-3-yl |
| A-147. | H | 2-methylpyridin-3-yl |
| A-148. | H | 2,4-dichloropyridin-3-yl |
| A-149. | H | 2,4-dibromopyridin-3-yl |
| A-150. | H | 2,4-difluoropyridin-3-yl |
| A-151. | H | 2-fluoro-4-chloropyridin-3-yl |
| A-152. | H | 2-chloro-4-fluoropyridin-3-yl |
| A-153. | H | 2-chloro-4-methylpyridin-3-yl |
| A-154. | H | 2-methyl-4-fluoropyridin-3-yl |
| A-155. | H | 2-methyl-4-chloropyridin-3-yl |
| A-156. | H | 2,4-dimethylpyridin-3-yl |
| A-157. | H | 2,4,6-trichloropyridin-3-yl |
| A-158. | H | 2,4,6-tribromopyridin-3-yl |
| A-159. | H | 2,4,6-trimethylpyridin-3-yl |
| A-160. | H | 2,4-dichloro-6-methylpyridin-3-yl |
| A-161. | H | 4-pyridyl |
| A-162. | H | 3-chloropyridin-4-yl |
| A-163. | H | 3-bromopyridin-4-yl |
| A-164. | H | 3-methylpyridin-4-yl |
| A-165. | H | 3,5-dichloropyridin-4-yl |
| A-166. | H | 3,5-dibromopyridin-4-yl |
| A-167. | H | 3,5-dimethylpyridin-4-yl |
| A-168. | H | 4-pyrimidinyl |
| A-169. | H | 5-chloropyrimidin-4-yl |
| A-170. | H | 5-fluoropyrimidin-4-yl |
| A-171. | H | 5-fluoro-6-chloropyrimidin-4-yl |
| A-172. | H | 2-methyl-6-trifluoromethylpyrimidin-4-yl |
| A-173. | H | 2,5-dimethyl-6-trifluoromethylpyrimidin-4-yl |
| A-174. | H | 5-methyl-6-trifluoromethylpyrimidin-4-yl |
| A-175. | H | 6-trifluoromethylpyrimidin-4-yl |
| A-176. | H | 2-methyl-5-fluoropyrimidin-4-yl |
| A-177. | H | 2-methyl-5-chloropyrimidin-4-yl |
| A-178. | H | 5-chloro-6-methylpyrimidin-4-yl |
| A-179. | H | 5-chloro-6-ethylpyrimidin-4-yl |
| A-180. | H | 5-chloro-6-isopropylpyrimidin-4-yl |
| A-181. | H | 5-bromo-6-methylpyrimidin-4-yl |
| A-182. | H | 5-fluoro-6-methylpyrimidin-4-yl |
| A-183. | H | 5-fluoro-6-fluoromethylpyrimidin-4-yl |
| A-184. | H | 2,6-dimethyl-5-chloropyrimidin-4-yl |
| A-185. | H | 5,6-dimethylpyrimidin-4-yl |
| A-186. | H | 2,5-dimethylpyrimidin-4-yl |
| A-187. | H | 2,5,6-trimethylpyrimidin-4-yl |
| A-188. | H | 5-methyl-6-methoxypyrimidin-4-yl |
| A-189. | H | 5-pyrimidinyl |
| A-190. | H | 4-methylpyrimidin-5-yl |
| A-191. | H | 4,6-dimethylpyrimidin-5-yl |
| A-192. | H | 2,4,6-trimethylpyrimidin-5-yl |
| A-193. | H | 4-trifluoromethyl-6-methylpyrimidin-5-yl |
| A-194. | H | 2-pyrimidinyl |
| A-195. | H | 4,6-dimethylpyrimidin-2-yl |
| A-196. | H | 4,5,6-trimethylpyrimidin-2-yl |
| A-197. | H | 4,6-ditrifluoromethylpyrimidin-2-yl |
| A-198. | H | 4,6-dimethyl-5-chloropyrimidin-2-yl |
| A-199. | CH₃ | phenyl |
| A-200. | CH₃ | 2-fluorophenyl |
| A-201. | CH₃ | 3-fluorophenyl |
| A-202. | CH₃ | 4-fluorophenyl |
| A-203. | CH₃ | 2-chlorophenyl |
| A-204. | CH₃ | 3-chlorophenyl |
| A-205. | CH₃ | 4-chlorophenyl |
| A-206. | CH₃ | 3-bromophenyl |
| A-207. | CH₃ | 4-bromophenyl |
| A-208. | CH₃ | 2-trifluoromethylphenyl |
| A-209. | CH₃ | 3-trifluoromethylphenyl |
| A-210. | CH₃ | 4-trifluoromethylphenyl |
| A-211. | CH₃ | 2-(methylthio)phenyl |
| A-212. | CH₃ | 3-(methylthio)phenyl |
| A-213. | CH₃ | 4-(methylthio)phenyl |
| A-214. | CH₃ | 2-methoxyphenyl |
| A-215. | CH₃ | 3-methoxyphenyl |
| A-216. | CH₃ | 4-methoxyphenyl |
| A-217. | CH₃ | 4-nitrophenyl |
| A-218. | CH₃ | 4-cyanophenyl |
| A-219. | CH₃ | 4-tert-butylphenyl |
| A-220. | CH₃ | 4-isopropylphenyl |
| A-221. | CH₃ | 3-ethoxyphenyl |
| A-222. | CH₃ | 4-ethoxyphenyl |
| A-223. | CH₃ | 4-n-propoxyphenyl |
| A-224. | CH₃ | 3-isopropoxyphenyl |
| A-225. | CH₃ | 4-isopropoxyphenyl |
| A-226. | CH₃ | 4-n-butoxyphenyl |
| A-227. | CH₃ | 4-tert-butoxyphenyl |
| A-228. | CH₃ | 4-(methoxyiminomethyl)phenyl |
| A-229. | CH₃ | 4-(1-(methoxyimino)ethyl)phenyl |
| A-230. | CH₃ | 2,3-difluorophenyl |
| A-231. | CH₃ | 2,4-difluorophenyl |
| A-232. | CH₃ | 2,5-difluorophenyl |
| A-233. | CH₃ | 3,4-difluorophenyl |
| A-234. | CH₃ | 3,5-difluorophenyl |
| A-235. | CH₃ | 2,6-difluorophenyl |
| A-236. | CH₃ | 2,4,6-trifluorophenyl |
| A-237. | CH₃ | 2,4,5-trifluorophenyl |
| A-238. | CH₃ | 2,3,4-trifluorophenyl |
| A-239. | CH₃ | 2,3,5-trifluorophenyl |
| A-240. | CH₃ | 3,4,5-trifluorophenyl |
| A-241. | CH₃ | 2,3-dichlorophenyl |
| A-242. | CH₃ | 2,5-dichlorophenyl |
| A-243. | CH₃ | 3,5-dichlorophenyl |
| A-244. | CH₃ | 2,6-dichlorophenyl |
| A-245. | CH₃ | 2,3-dimethylphenyl |
| A-246. | CH₃ | 2,4-dimethylphenyl |
| A-247. | CH₃ | 2,5-dimethylphenyl |
| A-248. | CH₃ | 2,4,5-trimethylphenyl |
| A-249. | CH₃ | 2,3-dimethoxyphenyl |
| A-250. | CH₃ | 2,4-dimethoxyphenyl |
| A-251. | CH₃ | 3,4-dimethoxyphenyl |
| A-252. | CH₃ | 2,4-bis(trifluoromethyl)phenyl |
| A-253. | CH₃ | 3,5-bis(trifluoromethyl)phenyl |
| A-254. | CH₃ | 2-methyl-3-methoxyphenyl |
| A-255. | CH₃ | 2-methyl-4-methoxyphenyl |
| A-256. | CH₃ | 2-methyl-6-methoxyphenyl |
| A-257. | CH₃ | 3-chloro-4-fluorophenyl |
| A-258. | CH₃ | 2-chloro-4-fluorophenyl |
| A-259. | CH₃ | 2-chloro-6-fluorophenyl |
| A-260. | CH₃ | 4-chloro-2-fluorophenyl |
| A-261. | CH₃ | 5-chloro-2-fluorophenyl |
| A-262. | CH₃ | 4-fluoro-3-methylphenyl |
| A-263. | CH₃ | 2-fluoro-4-methylphenyl |
| A-264. | CH₃ | 4-fluoro-2-methylphenyl |
| A-265. | CH₃ | 2-fluoro-3-methoxyphenyl |
| A-266. | CH₃ | 2-fluoro-4-methoxyphenyl |
| A-267. | CH₃ | 2-fluoro-6-methoxyphenyl |
| A-268. | CH₃ | 2-fluoro-4-trifluoromethylphenyl |
| A-269. | CH₃ | 4-chloro-3-methylphenyl |
| A-270. | CH₃ | 2-chloro-4-methylphenyl |
| A-271. | CH₃ | 2-chloro-6-methylphenyl |
| A-272. | CH₃ | 5-chloro-2-methylphenyl |
| A-273. | CH₃ | 3-chloro-2-methylphenyl |
| A-274. | CH₃ | 2-chloro-4-methoxyphenyl |
| A-275. | CH₃ | 2-chloro-6-methoxyphenyl |
| A-276. | CH₃ | 2-chloro-4-trifluoromethylphenyl |
| A-277. | CH₃ | 3-fluoro-4-methylphenyl |
| A-278. | CH₃ | 3-fluoro-4-isopropylphenyl |
| A-279. | CH₃ | 4-fluoro-3-methylphenyl |
| A-280. | CH₃ | 3-fluoro-4-methoxyphenyl |
| A-281. | CH₃ | 3-fluoro-4-ethoxyphenyl |
| A-282. | CH₃ | 3-fluoro-4-trifluoromethylphenyl |
| A-283. | CH₃ | 3-chloro-4-methylphenyl |
| A-284. | CH₃ | 3-chloro-4-methoxyphenyl |
| A-285. | CH₃ | 3-chloro-4-ethoxyphenyl |
| A-286. | CH₃ | 3-chloro-4-trifluoromethylphenyl |
| A-287. | CH₃ | 3-methyl-4-methoxyphenyl |
| A-288. | CH₃ | 4-chloro-2,5-difluorophenyl |
| A-289. | CH₃ | 4-tert-butyl-2-fluorophenyl |
| A-290. | CH₃ | 2-fluoro-4-isopropylphenyl |
| A-291. | CH₃ | 4-ethoxy-2-fluorophenyl |
| A-292. | CH₃ | 4-acetyl-2-fluorophenyl |
| A-293. | CH₃ | 2-thienyl |
| A-294. | CH₃ | 5-methylthiophen-2-yl |
| A-295. | CH₃ | 4-methylthiophen-2-yl |
| A-296. | CH₃ | 5-chlorothiophen-2-yl |
| A-297. | CH₃ | 3-cyanothiophen-2-yl |
| A-298. | CH₃ | 5-formylthiophen-2-yl |
| A-299. | CH₃ | 5-acetylthiophen-2-yl |
| A-300. | CH₃ | 5-(methoxyiminomethyl)thiophen-2-yl |
| A-301. | CH₃ | 5-(1-(methoxyimino)ethyl)thiophen-2-yl |
| A-302. | CH₃ | 4-bromothiophen-2-yl |
| A-303. | CH₃ | 3,5-dichlorothiophen-2-yl |
| A-304. | CH₃ | 3-thienyl |
| A-305. | CH₃ | 2-methylthiophen-3-yl |
| A-306. | CH₃ | 2,5-dichlorothiophen-3-yl |
| A-307. | CH₃ | 2,4,5-trichlorothiophen-3-yl |
| A-308. | CH₃ | 2-furyl |
| A-309. | CH₃ | 5-methylfuran-2-yl |
| A-310. | CH₃ | 5-chlorofuran-2-yl |
| A-311. | CH₃ | 4-methylfuran-2-yl |
| A-312. | CH₃ | 3-cyanofuran-2-yl |
| A-313. | CH₃ | 5-acetylfuran-2-yl |
| A-314. | CH₃ | 3-furyl |
| A-315. | CH₃ | 2-methylfuran-3-yl |
| A-316. | CH₃ | 2,5-dimethylfuran-3-yl |
| A-317. | CH₃ | 2-pyridyl |
| A-318. | CH₃ | 3-fluoropyridin-2-yl |
| A-319. | CH₃ | 3-chloropyridin-2-yl |
| A-320. | CH₃ | 3-bromopyridin-2-yl |
| A-321. | CH₃ | 3-trifluoromethylpyridin-2-yl |
| A-322. | CH₃ | 3-methylpyridin-2-yl |
| A-323. | CH₃ | 3-ethylpyridin-2-yl |
| A-324. | CH₃ | 3,5-difluoropyridin-2-yl |
| A-325. | CH₃ | 3,5-dichloropyridin-2-yl |
| A-326. | CH₃ | 3,5-dibromopyridin-2-yl |
| A-327. | CH₃ | 3,5-dimethylpyridin-2-yl |
| A-328. | CH₃ | 3-fluoro-5-trifluoromethylpyridin-2-yl |
| A-329. | CH₃ | 3-chloro-5-fluoropyridin-2-yl |
| A-330. | CH₃ | 3-chloro-5-methylpyridin-2-yl |
| A-331. | CH₃ | 3-fluoro-5-chloropyridin-2-yl |
| A-332. | CH₃ | 3-fluoro-5-methylpyridin-2-yl |
| A-333. | CH₃ | 3-methyl-5-fluoropyridin-2-yl |
| A-334. | CH₃ | 3-methyl-5-chloropyridin-2-yl |
| A-335. | CH₃ | 5-nitropyridin-2-yl |
| A-336. | CH₃ | 5-cyanopyridin-2-yl |
| A-337. | CH₃ | 5-methoxycarbonylpyridin-2-yl |
| A-338. | CH₃ | 5-trifluoromethylpyridin-2-yl |
| A-339. | CH₃ | 5-methylpyridin-2-yl |
| A-340. | CH₃ | 4-methylpyridin-2-yl |
| A-341. | CH₃ | 6-methylpyridin-2-yl |
| A-342. | CH₃ | 3-pyridyl |
| A-343. | CH₃ | 2-chloropyridin-3-yl |
| A-344. | CH₃ | 2-bromopyridin-3-yl |
| A-345. | CH₃ | 2-methylpyridin-3-yl |
| A-346. | CH₃ | 2,4-dichloropyridin-3-yl |
| A-347. | CH₃ | 2,4-dibromopyridin-3-yl |
| A-348. | CH₃ | 2,4-difluoropyridin-3-yl |
| A-349. | CH₃ | 2-fluoro-4-chloropyridin-3-yl |
| A-350. | CH₃ | 2-chloro-4-fluoropyridin-3-yl |
| A-351. | CH₃ | 2-chloro-4-methylpyridin-3-yl |
| A-352. | CH₃ | 2-methyl-4-fluoropyridin-3-yl |
| A-353. | CH₃ | 2-methyl-4-chloropyridin-3-yl |
| A-354. | CH₃ | 2,4-dimethylpyridin-3-yl |
| A-355. | CH₃ | 2,4,6-trichloropyridin-3-yl |
| A-356. | CH₃ | 2,4,6-tribromopyridin-3-yl |
| A-357. | CH₃ | 2,4,6-trimethylpyridin-3-yl |
| A-358. | CH₃ | 2,4-dichloro-6-methylpyridin-3-yl |
| A-359. | CH₃ | 4-pyridyl |
| A-360. | CH₃ | 3-chloropyridin-4-yl |
| A-361. | CH₃ | 3-bromopyridin-4-yl |
| A-362. | CH₃ | 3-methylpyridin-4-yl |
| A-363. | CH₃ | 3,5-dichloropyridin-4-yl |
| A-364. | CH₃ | 3,5-dibromopyridin-4-yl |
| A-365. | CH₃ | 3,5-dimethylpyridin-4-yl |
| A-366. | CH₃ | 4-pyrimidinyl |
| A-367. | CH₃ | 5-chloropyrimidin-4-yl |
| A-368. | CH₃ | 5-fluoropyrimidin-4-yl |
| A-369. | CH₃ | 5-fluoro-6-chloropyrimidin-4-yl |
| A-370. | CH₃ | 2-methyl-6-trifluoromethylpyrimidin-4-yl |
| A-371. | CH₃ | 2,5-dimethyl-6-trifluoromethylpyrimidin-4-yl |
| A-372. | CH₃ | 5-methyl-6-trifluoromethylpyrimidin-4-yl |
| A-373. | CH₃ | 6-trifluoromethylpyrimidin-4-yl |
| A-374. | CH₃ | 2-methyl-5-fluoropyrimidin-4-yl |
| A-375. | CH₃ | 2-methyl-5-chloropyrimidin-4-yl |
| A-376. | CH₃ | 5-chloro-6-methylpyrimidin-4-yl |
| A-377. | CH₃ | 5-chloro-6-ethylpyrimidin-4-yl |
| A-378. | CH₃ | 5-chloro-6-isopropylpyrimidin-4-yl |
| A-379. | CH₃ | 5-bromo-6-methylpyrimidin-4-yl |
| A-380. | CH₃ | 5-fluoro-6-methylpyrimidin-4-yl |
| A-381. | CH₃ | 5-fluoro-6-fluoromethylpyrimidin-4-yl |
| A-382. | CH₃ | 2,6-dimethyl-5-chloropyrimidin-4-yl |
| A-383. | CH₃ | 5,6-dimethylpyrimidin-4-yl |
| A-384. | CH₃ | 2,5-dimethylpyrimidin-4-yl |
| A-385. | CH₃ | 2,5,6-trimethylpyrimidin-4-yl |
| A-386. | CH₃ | 5-methyl-6-methoxypyrimidin-4-yl |
| A-387. | CH₃ | 5-pyrimidinyl |
| A-388. | CH₃ | 4-methylpyrimidin-5-yl |
| A-389. | CH₃ | 4,6-dimethylpyrimidin-5-yl |
| A-390. | CH₃ | 2,4,6-trimethylpyrimidin-5-yl |
| A-391. | CH₃ | 4-trifluoromethyl-6-methylpyrimidin-5-yl |
| A-392. | CH₃ | 2-pyrimidinyl |
| A-393. | CH₃ | 4,6-dimethylpyrimidin-2-yl |
| A-394. | CH₃ | 4,5,6-trimethylpyrimidin-2-yl |
| A-395. | CH₃ | 4,6-ditrifluoromethylpyrimidin-2-yl |
| A-396. | CH₃ | 4,6-dimethyl-5-chloropyrimidin-2-yl |
| A-397. | CF₃ | phenyl |
| A-398. | CF₃ | 2-fluorophenyl |
| A-399. | CF₃ | 3-fluorophenyl |
| A-400. | CF₃ | 4-fluorophenyl |
| A-401. | CF₃ | 2-chlorophenyl |
| A-402. | CF₃ | 3-chlorophenyl |
| A-403. | CF₃ | 4-chlorophenyl |
| A-404. | CF₃ | 3-bromophenyl |
| A-405. | CF₃ | 4-bromophenyl |
| A-406. | CF₃ | 2-trifluoromethylphenyl |
| A-407. | CF₃ | 3-trifluoromethylphenyl |
| A-408. | CF₃ | 4-trifluoromethylphenyl |
| A-409. | CF₃ | 2-(methylthio)phenyl |
| A-410. | CF₃ | 3-(methylthio)phenyl |
| A-411. | CF₃ | 4-(methylthio)phenyl |
| A-412. | CF₃ | 2-methoxyphenyl |
| A-413. | CF₃ | 3-methoxyphenyl |
| A-414. | CF₃ | 4-methoxyphenyl |
| A-415. | CF₃ | 4-nitrophenyl |
| A-416. | CF₃ | 4-cyanophenyl |
| A-417. | CF₃ | 4-tert-butylphenyl |
| A-418. | CF₃ | 4-isopropylphenyl |
| A-419. | CF₃ | 3-ethoxyphenyl |
| A-420. | CF₃ | 4-ethoxyphenyl |
| A-421. | CF₃ | 4-n-propoxyphenyl |
| A-422. | CF₃ | 3-isopropoxyphenyl |
| A-423. | CF₃ | 4-isopropoxyphenyl |
| A-424. | CF₃ | 4-n-butoxyphenyl |
| A-425. | CF₃ | 4-tert-butoxyphenyl |
| A-426. | CF₃ | 4-(methoxyiminomethyl)phenyl |
| A-427. | CF₃ | 4-(1-(methoxyimino)ethyl)phenyl |
| A-428. | CF₃ | 2,3-difluorophenyl |
| A-429. | CF₃ | 2,4-difluorophenyl |
| A-430. | CF₃ | 2,5-difluorophenyl |
| A-431. | CF₃ | 3,4-difluorophenyl |
| A-432. | CF₃ | 3,5-difluorophenyl |
| A-433. | CF₃ | 2,6-difluorophenyl |
| A-434. | CF₃ | 2,4,6-trifluorophenyl |
| A-435. | CF₃ | 2,4,5-trifluorophenyl |
| A-436. | CF₃ | 2,3,4-trifluorophenyl |
| A-437. | CF₃ | 2,3,5-trifluorophenyl |
| A-438. | CF₃ | 3,4,5-trifluorophenyl |
| A-439. | CF₃ | 2,3-dichlorophenyl |
| A-440. | CF₃ | 2,5-dichlorophenyl |
| A-441. | CF₃ | 3,5-dichlorophenyl |
| A-442. | CF₃ | 2,6-dichlorophenyl |
| A-443. | CF₃ | 2,3-dimethylphenyl |
| A-444. | CF₃ | 2,4-dimethylphenyl |
| A-445. | CF₃ | 2,5-dimethylphenyl |
| A-446. | CF₃ | 2,4,5-trimethylphenyl |
| A-447. | CF₃ | 2,3-dimethoxyphenyl |
| A-448. | CF₃ | 2,4-dimethoxyphenyl |
| A-449. | CF₃ | 3,4-dimethoxyphenyl |
| A-450. | CF₃ | 2,4-bis(trifluoromethyl)phenyl |
| A-451. | CF₃ | 3,5-bis(trifluoromethyl)phenyl |
| A-452. | CF₃ | 2-methyl-3-methoxyphenyl |
| A-453. | CF₃ | 2-methyl-4-methoxyphenyl |
| A-454. | CF₃ | 2-methyl-6-methoxyphenyl |
| A-455. | CF₃ | 3-chloro-4-fluorophenyl |
| A-456. | CF₃ | 2-chloro-4-fluorophenyl |
| A-457. | CF₃ | 2-chloro-6-fluorophenyl |
| A-458. | CF₃ | 4-chloro-2-fluorophenyl |
| A-459. | CF₃ | 5-chloro-2-fluorophenyl |
| A-460. | CF₃ | 4-fluoro-3-methylphenyl |
| A-461. | CF₃ | 2-fluoro-4-methylphenyl |
| A-462. | CF₃ | 4-fluoro-2-methylphenyl |
| A-463. | CF₃ | 2-fluoro-3-methoxyphenyl |
| A-464. | CF₃ | 2-fluoro-4-methoxyphenyl |
| A-465. | CF₃ | 2-fluoro-6-methoxyphenyl |
| A-466. | CF₃ | 2-fluoro-4-trifluoromethylphenyl |
| A-467. | CF₃ | 4-chloro-3-methylphenyl |
| A-468. | CF₃ | 2-chloro-4-methylphenyl |
| A-469. | CF₃ | 2-chloro-6-methylphenyl |
| A-470. | CF₃ | 5-chloro-2-methylphenyl |
| A-471. | CF₃ | 3-chloro-2-methylphenyl |
| A-472. | CF₃ | 2-chloro-4-methoxyphenyl |
| A-473. | CF₃ | 2-chloro-6-methoxyphenyl |
| A-474. | CF₃ | 2-chloro-4-trifluoromethylphenyl |
| A-475. | CF₃ | 3-fluoro-4-methylphenyl |
| A-476. | CF₃ | 3-fluoro-4-isopropylphenyl |
| A-477. | CF₃ | 4-fluoro-3-methylphenyl |
| A-478. | CF₃ | 3-fluoro-4-methoxyphenyl |
| A-479. | CF₃ | 3-fluoro-4-ethoxyphenyl |
| A-480. | CF₃ | 3-fluoro-4-trifluoromethylphenyl |
| A-481. | CF₃ | 3-chloro-4-methylphenyl |
| A-482. | CF₃ | 3-chloro-4-methoxyphenyl |
| A-483. | CF₃ | 3-chloro-4-ethoxyphenyl |
| A-484. | CF₃ | 3-chloro-4-trifluoromethylphenyl |
| A-485. | CF₃ | 3-methyl-4-methoxyphenyl |
| A-486. | CF₃ | 4-chloro-2,5-difluorophenyl |
| A-487. | CF₃ | 4-tert-butyl-2-fluorophenyl |
| A-488. | CF₃ | 2-fluoro-4-isopropylphenyl |
| A-489. | CF₃ | 4-ethoxy-2-fluorophenyl |
| A-490. | CF₃ | 4-acetyl-2-fluorophenyl |
| A-491. | CF₃ | 2-thienyl |
| A-492. | CF₃ | 5-methylthiophen-2-yl |
| A-493. | CF₃ | 4-methylthiophen-2-yl |
| A-494. | CF₃ | 5-chlorothiophen-2-yl |
| A-495. | CF₃ | 3-cyanothiophen-2-yl |
| A-496. | CF₃ | 5-formylthiophen-2-yl |
| A-497. | CF₃ | 5-acetylthiophen-2-yl |
| A-498. | CF₃ | 5-(methoxyiminomethyl)thiophen-2-yl |
| A-499. | CF₃ | 5-(1-(methoxyimino)ethyl)thiophen-2-yl |
| A-500. | CF₃ | 4-bromothiophen-2-yl |
| A-501. | CF₃ | 3,5-dichlorothiophen-2-yl |
| A-502. | CF₃ | 3-thienyl |
| A-503. | CF₃ | 2-methylthiophen-3-yl |
| A-504. | CF₃ | 2,5-dichlorothiophen-3-yl |
| A-505. | CF₃ | 2,4,5-trichlorothiophen-3-yl |
| A-506. | CF₃ | 2-furyl |
| A-507. | CF₃ | 5-methylfuran-2-yl |
| A-508. | CF₃ | 5-chlorofuran-2-yl |
| A-509. | CF₃ | 4-methylfuran-2-yl |
| A-510. | CF₃ | 3-cyanofuran-2-yl |
| A-511. | CF₃ | 5-acetylfuran-2-yl |
| A-512. | CF₃ | 3-furyl |
| A-513. | CF₃ | 2-methylfuran-3-yl |
| A-514. | CF₃ | 2,5-dimethylfuran-3-yl |
| A-515. | CF₃ | 2-pyridyl |
| A-516. | CF₃ | 3-fluoropyridin-2-yl |
| A-517. | CF₃ | 3-chloropyridin-2-yl |
| A-518. | CF₃ | 3-bromopyridin-2-yl |
| A-519. | CF₃ | 3-trifluoromethylpyridin-2-yl |
| A-520. | CF₃ | 3-methylpyridin-2-yl |
| A-521. | CF₃ | 3-ethylpyridin-2-yl |
| A-522. | CF₃ | 3,5-difluoropyridin-2-yl |
| A-523. | CF₃ | 3,5-dichloropyridin-2-yl |
| A-524. | CF₃ | 3,5-dibromopyridin-2-yl |
| A-525. | CF₃ | 3,5-dimethylpyridin-2-yl |
| A-526. | CF₃ | 3-fluoro-5-trifluoromethylpyridin-2-yl |
| A-527. | CF₃ | 3-chloro-5-fluoropyridin-2-yl |
| A-528. | CF₃ | 3-chloro-5-methylpyridin-2-yl |
| A-529. | CF₃ | 3-fluoro-5-chloropyridin-2-yl |
| A-530. | CF₃ | 3-fluoro-5-methylpyridin-2-yl |
| A-531. | CF₃ | 3-methyl-5-fluoropyridin-2-yl |
| A-532. | CF₃ | 3-methyl-5-chloropyridin-2-yl |
| A-533. | CF₃ | 5-nitropyridin-2-yl |
| A-534. | CF₃ | 5-cyanopyridin-2-yl |
| A-535. | CF₃ | 5-methoxycarbonylpyridin-2-yl |
| A-536. | CF₃ | 5-trifluoromethylpyridin-2-yl |
| A-537. | CF₃ | 5-methylpyridin-2-yl |
| A-538. | CF₃ | 4-methylpyridin-2-yl |
| A-539. | CF₃ | 6-methylpyridin-2-yl |
| A-540. | CF₃ | 3-pyridyl |
| A-541. | CF₃ | 2-chloropyridin-3-yl |
| A-542. | CF₃ | 2-bromopyridin-3-yl |
| A-543. | CF₃ | 2-methylpyridin-3-yl |
| A-544. | CF₃ | 2,4-dichloropyridin-3-yl |
| A-545. | CF₃ | 2,4-dibromopyridin-3-yl |
| A-546. | CF₃ | 2,4-difluoropyridin-3-yl |
| A-547. | CF₃ | 2-fluoro-4-chloropyridin-3-yl |
| A-548. | CF₃ | 2-chloro-4-fluoropyridin-3-yl |
| A-549. | CF₃ | 2-chloro-4-methylpyridin-3-yl |
| A-550. | CF₃ | 2-methyl-4-fluoropyridin-3-yl |
| A-551. | CF₃ | 2-methyl-4-chloropyridin-3-yl |
| A-552. | CF₃ | 2,4-dimethylpyridin-3-yl |
| A-553. | CF₃ | 2,4,6-trichloropyridin-3-yl |
| A-554. | CF₃ | 2,4,6-tribromopyridin-3-yl |
| A-555. | CF₃ | 2,4,6-trimethylpyridin-3-yl |
| A-556. | CF₃ | 2,4-dichloro-6-methylpyridin-3-yl |
| A-557. | CF₃ | 4-pyridyl |
| A-558. | CF₃ | 3-chloropyridin-4-yl |
| A-559. | CF₃ | 3-bromopyridin-4-yl |
| A-560. | CF₃ | 3-methylpyridin-4-yl |
| A-561. | CF₃ | 3,5-dichloropyridin-4-yl |
| A-562. | CF₃ | 3,5-dibromopyridin-4-yl |
| A-563. | CF₃ | 3,5-dimethylpyridin-4-yl |
| A-564. | CF₃ | 4-pyrimidinyl |
| A-565. | CF₃ | 5-chloropyrimidin-4-yl |
| A-566. | CF₃ | 5-fluoropyrimidin-4-yl |
| A-567. | CF₃ | 5-fluoro-6-chloropyrimidin-4-yl |
| A-568. | CF₃ | 2-methyl-6-trifluoromethylpyrimidin-4-yl |
| A-569. | CF₃ | 2,5-dimethyl-6-trifluoromethylpyrimidin-4-yl |
| A-570. | CF₃ | 5-methyl-6-trifluoromethylpyrimidin-4-yl |
| A-571. | CF₃ | 6-trifluoromethylpyrimidin-4-yl |
| A-572. | CF₃ | 2-methyl-5-fluoropyrimidin-4-yl |
| A-573. | CF₃ | 2-methyl-5-chloropyrimidin-4-yl |
| A-574. | CF₃ | 5-chloro-6-methylpyrimidin-4-yl |
| A-575. | CF₃ | 5-chloro-6-ethylpyrimidin-4-yl |
| A-576. | CF₃ | 5-chloro-6-isopropylpyrimidin-4-yl |
| A-577. | CF₃ | 5-bromo-6-methylpyrimidin-4-yl |
| A-578. | CF₃ | 5-fluoro-6-methylpyrimidin-4-yl |
| A-579. | CF₃ | 5-fluoro-6-fluoromethylpyrimidin-4-yl |
| A-580. | CF₃ | 2,6-dimethyl-5-chloropyrimidin-4-yl |
| A-581. | CF₃ | 5,6-dimethylpyrimidin-4-yl |
| A-582. | CF₃ | 2,5-dimethylpyrimidin-4-yl |
| A-583. | CF₃ | 2,5,6-trimethylpyrimidin-4-yl |
| A-584. | CF₃ | 5-methyl-6-methoxypyrimidin-4-yl |
| A-585. | CF₃ | 5-pyrimidinyl |
| A-586. | CF₃ | 4-methylpyrimidin-5-yl |
| A-587. | CF₃ | 4,6-dimethylpyrimidin-5-yl |
| A-588. | CF₃ | 2,4,6-trimethylpyrimidin-5-yl |
| A-589. | CF₃ | 4-trifluoromethyl-6-methylpyrimidin-5-yl |
| A-590. | CF₃ | 2-pyrimidinyl |
| A-591. | CF₃ | 4,6-dimethylpyrimidin-2-yl |
| A-592. | CF₃ | 4,5,6-trimethylpyrimidin-2-yl |
| A-593. | CF₃ | 4,6-ditrifluoromethylpyrimidin-2-yl |
| A-594. | CF₃ | 4,6-dimethyl-5-chloropyrimidin-2-yl |
| A-595. | OCH₃ | phenyl |
| A-596. | OCH₃ | 2-fluorophenyl |
| A-597. | OCH₃ | 3-fluorophenyl |
| A-598. | OCH₃ | 4-fluorophenyl |
| A-599. | OCH₃ | 2-chlorophenyl |
| A-600. | OCH₃ | 3-chlorophenyl |
| A-601. | OCH₃ | 4-chlorophenyl |
| A-602. | OCH₃ | 3-bromophenyl |
| A-603. | OCH₃ | 4-bromophenyl |
| A-604. | OCH₃ | 2-trifluoromethylphenyl |
| A-605. | OCH₃ | 3-trifluoromethylphenyl |
| A-606. | OCH₃ | 4-trifluoromethylphenyl |
| A-607. | OCH₃ | 2-(methylthio)phenyl |
| A-608. | OCH₃ | 3-(methylthio)phenyl |
| A-609. | OCH₃ | 4-(methylthio)phenyl |
| A-610. | OCH₃ | 2-methoxyphenyl |
| A-611. | OCH₃ | 3-methoxyphenyl |
| A-612. | OCH₃ | 4-methoxyphenyl |
| A-613. | OCH₃ | 4-nitrophenyl |
| A-614. | OCH₃ | 4-cyanophenyl |
| A-615. | OCH₃ | 4-tert-butylphenyl |
| A-616. | OCH₃ | 4-isopropylphenyl |
| A-617. | OCH₃ | 3-ethoxyphenyl |
| A-618. | OCH₃ | 4-ethoxyphenyl |
| A-619. | OCH₃ | 4-n-propoxyphenyl |
| A-620. | OCH₃ | 3-isopropoxyphenyl |
| A-621. | OCH₃ | 4-isopropoxyphenyl |
| A-622. | OCH₃ | 4-n-butoxyphenyl |
| A-623. | OCH₃ | 4-tert-butoxyphenyl |
| A-624. | OCH₃ | 4-(methoxyiminomethyl)phenyl |
| A-625. | OCH₃ | 4-(1-(methoxyimino)ethyl)phenyl |
| A-626. | OCH₃ | 2,3-difluorophenyl |
| A-627. | OCH₃ | 2,4-difluorophenyl |
| A-628. | OCH₃ | 2,5-difluorophenyl |
| A-629. | OCH₃ | 3,4-difluorophenyl |
| A-630. | OCH₃ | 3,5-difluorophenyl |
| A-631. | OCH₃ | 2,6-difluorophenyl |
| A-632. | OCH₃ | 2,4,6-trifluorophenyl |
| A-633. | OCH₃ | 2,4,5-trifluorophenyl |
| A-634. | OCH₃ | 2,3,4-trifluorophenyl |
| A-635. | OCH₃ | 2,3,5-trifluorophenyl |
| A-636. | OCH₃ | 3,4,5-trifluorophenyl |
| A-637. | OCH₃ | 2,3-dichlorophenyl |
| A-638. | OCH₃ | 2,5-dichlorophenyl |
| A-639. | OCH₃ | 3,5-dichlorophenyl |
| A-640. | OCH₃ | 2,6-dichlorophenyl |
| A-641. | OCH₃ | 2,3-dimethylphenyl |
| A-642. | OCH₃ | 2,4-dimethylphenyl |
| A-643. | OCH₃ | 2,5-dimethylphenyl |
| A-644. | OCH₃ | 2,4,5-trimethylphenyl |
| A-645. | OCH₃ | 2,3-dimethoxyphenyl |
| A-646. | OCH₃ | 2,4-dimethoxyphenyl |
| A-647. | OCH₃ | 3,4-dimethoxyphenyl |
| A-648. | OCH₃ | 2,4-bis(trifluoromethyl)phenyl |
| A-649. | OCH₃ | 3,5-bis(trifluoromethyl)phenyl |
| A-650. | OCH₃ | 2-methyl-3-methoxyphenyl |
| A-651. | OCH₃ | 2-methyl-4-methoxyphenyl |
| A-652. | OCH₃ | 2-methyl-6-methoxyphenyl |
| A-653. | OCH₃ | 3-chloro-4-fluorophenyl |
| A-654. | OCH₃ | 2-chloro-4-fluorophenyl |
| A-655. | OCH₃ | 2-chloro-6-fluorophenyl |
| A-656. | OCH₃ | 4-chloro-2-fluorophenyl |
| A-657. | OCH₃ | 5-chloro-2-fluorophenyl |
| A-658. | OCH₃ | 4-fluoro-3-methylphenyl |
| A-659. | OCH₃ | 2-fluoro-4-methylphenyl |
| A-660. | OCH₃ | 4-fluoro-2-methylphenyl |
| A-661. | OCH₃ | 2-fluoro-3-methoxyphenyl |
| A-662. | OCH₃ | 2-fluoro-4-methoxyphenyl |
| A-663. | OCH₃ | 2-fluoro-6-methoxyphenyl |
| A-664. | OCH₃ | 2-fluoro-4-trifluoromethylphenyl |
| A-665. | OCH₃ | 4-chloro-3-methylphenyl |
| A-666. | OCH₃ | 2-chloro-4-methylphenyl |
| A-667. | OCH₃ | 2-chloro-6-methylphenyl |
| A-668. | OCH₃ | 5-chloro-2-methylphenyl |
| A-669. | OCH₃ | 3-chloro-2-methylphenyl |
| A-670. | OCH₃ | 2-chloro-4-methoxyphenyl |
| A-671. | OCH₃ | 2-chloro-6-methoxyphenyl |
| A-672. | OCH₃ | 2-chloro-4-trifluoromethylphenyl |
| A-673. | OCH₃ | 3-fluoro-4-methylphenyl |
| A-674. | OCH₃ | 3-fluoro-4-isopropylphenyl |
| A-675. | OCH₃ | 4-fluoro-3-methylphenyl |
| A-676. | OCH₃ | 3-fluoro-4-methoxyphenyl |
| A-677. | OCH₃ | 3-fluoro-4-ethoxyphenyl |
| A-678. | OCH₃ | 3-fluoro-4-trifluoromethylphenyl |
| A-679. | OCH₃ | 3-chloro-4-methylphenyl |
| A-680. | OCH₃ | 3-chloro-4-methoxyphenyl |
| A-681. | OCH₃ | 3-chloro-4-ethoxyphenyl |
| A-682. | OCH₃ | 3-chloro-4-trifluoromethylphenyl |
| A-683. | OCH₃ | 3-methyl-4-methoxyphenyl |
| A-684. | OCH₃ | 4-chloro-2,5-difluorophenyl |
| A-685. | OCH₃ | 4-tert-butyl-2-fluorophenyl |
| A-686. | OCH₃ | 2-fluoro-4-isopropylphenyl |
| A-687. | OCH₃ | 4-ethoxy-2-fluorophenyl |
| A-688. | OCH₃ | 4-acetyl-2-fluorophenyl |
| A-689. | OCH₃ | 2-thienyl |
| A-690. | OCH₃ | 5-methylthiophen-2-yl |
| A-691. | OCH₃ | 4-methylthiophen-2-yl |
| A-692. | OCH₃ | 5-chlorothiophen-2-yl |
| A-693. | OCH₃ | 3-cyanothiophen-2-yl |
| A-694. | OCH₃ | 5-formylthiophen-2-yl |
| A-695. | OCH₃ | 5-acetylthiophen-2-yl |
| A-696. | OCH₃ | 5-(methoxyiminomethyl)thiophen-2-yl |
| A-697. | OCH₃ | 5-(1-(methoxyimino)ethyl)thiophen-2-yl |
| A-698. | OCH₃ | 4-bromothiophen-2-yl |
| A-699. | OCH₃ | 3,5-dichlorothiophen-2-yl |
| A-700. | OCH₃ | 3-thienyl |
| A-701. | OCH₃ | 2-methylthiophen-3-yl |
| A-702. | OCH₃ | 2,5-dichlorothiophen-3-yl |
| A-703. | OCH₃ | 2,4,5-trichloro-thiophen-3-yl |
| A-704. | OCH₃ | 2-furyl |
| A-705. | OCH₃ | 5-methylfuran-2-yl |
| A-706. | OCH₃ | 5-chlorofuran-2-yl |
| A-707. | OCH₃ | 4-methylfuran-2-yl |
| A-708. | OCH₃ | 3-cyanofuran-2-yl |
| A-709. | OCH₃ | 5-acetylfuran-2-yl |
| A-710. | OCH₃ | 3-furyl |
| A-711. | OCH₃ | 2-methylfuran-3-yl |
| A-712. | OCH₃ | 2,5-dimethylfuran-3-yl |
| A-713. | OCH₃ | 2-pyridyl |
| A-714. | OCH₃ | 3-fluoropyridin-2-yl |
| A-715. | OCH₃ | 3-chloropyridin-2-yl |
| A-716. | OCH₃ | 3-bromopyridin-2-yl |
| A-717. | OCH₃ | 3-trifluoromethylpyridin-2-yl |
| A-718. | OCH₃ | 3-methylpyridin-2-yl |
| A-719. | OCH₃ | 3-ethylpyridin-2-yl |
| A-720. | OCH₃ | 3,5-difluoropyridin-2-yl |
| A-721. | OCH₃ | 3,5-dichloropyridin-2-yl |
| A-722. | OCH₃ | 3,5-dibromopyridin-2-yl |
| A-723. | OCH₃ | 3,5-dimethylpyridin-2-yl |
| A-724. | OCH₃ | 3-fluoro-5-trifluoromethylpyridin-2-yl |
| A-725. | OCH₃ | 3-chloro-5-fluoropyridin-2-yl |
| A-726. | OCH₃ | 3-chloro-5-methylpyridin-2-yl |
| A-727. | OCH₃ | 3-fluoro-5-chloropyridin-2-yl |
| A-728. | OCH₃ | 3-fluoro-5-methylpyridin-2-yl |
| A-729. | OCH₃ | 3-methyl-5-fluoropyridin-2-yl |
| A-730. | OCH₃ | 3-methyl-5-chloropyridin-2-yl |
| A-731. | OCH₃ | 5-nitropyridin-2-yl |
| A-732. | OCH₃ | 5-cyanopyridin-2-yl |
| A-733. | OCH₃ | 5-methoxycarbonylpyridin-2-yl |
| A-734. | OCH₃ | 5-trifluoromethylpyridin-2-yl |
| A-735. | OCH₃ | 5-methylpyridin-2-yl |
| A-736. | OCH₃ | 4-methylpyridin-2-yl |
| A-737. | OCH₃ | 6-methylpyridin-2-yl |
| A-738. | OCH₃ | 3-pyridyl |
| A-739. | OCH₃ | 2-chloropyridin-3-yl |
| A-740. | OCH₃ | 2-bromopyridin-3-yl |
| A-741. | OCH₃ | 2-methylpyridin-3-yl |
| A-742. | OCH₃ | 2,4-dichloropyridin-3-yl |
| A-743. | OCH₃ | 2,4-dibromopyridin-3-yl |
| A-744. | OCH₃ | 2,4-difluoropyridin-3-yl |
| A-745. | OCH₃ | 2-fluoro-4-chloropyridin-3-yl |
| A-746. | OCH₃ | 2-chloro-4-fluoropyridin-3-yl |
| A-747. | OCH₃ | 2-chloro-4-methylpyridin-3-yl |
| A-748. | OCH₃ | 2-methyl-4-fluoropyridin-3-yl |
| A-749. | OCH₃ | 2-methyl-4-chloropyridin-3-yl |
| A-750. | OCH₃ | 2,4-dimethylpyridin-3-yl |
| A-751. | OCH₃ | 2,4,6-trichloropyridin-3-yl |
| A-752. | OCH₃ | 2,4,6-tribromopyridin-3-yl |
| A-753. | OCH₃ | 2,4,6-trimethylpyridin-3-yl |
| A-754. | OCH₃ | 2,4-dichloro-6-methylpyridin-3-yl |
| A-755. | OCH₃ | 4-pyridyl |
| A-756. | OCH₃ | 3-chloropyridin-4-yl |
| A-757. | OCH₃ | 3-bromopyridin-4-yl |
| A-758. | OCH₃ | 3-methylpyridin-4-yl |
| A-759. | OCH₃ | 3,5-dichloropyridin-4-yl |
| A-760. | OCH₃ | 3,5-dibromopyridin-4-yl |
| A-761. | OCH₃ | 3,5-dimethylpyridin-4-yl |
| A-762. | OCH₃ | 4-pyrimidinyl |
| A-763. | OCH₃ | 5-chloropyrimidin-4-yl |
| A-764. | OCH₃ | 5-fluoropyrimidin-4-yl |
| A-765. | OCH₃ | 5-fluoro-6-chloropyrimidin-4-yl |
| A-766. | OCH₃ | 2-methyl-6-trifluoromethylpyrimidin-4-yl |
| A-767. | OCH₃ | 2,5-dimethyl-6-trifluoromethylpyrimidin-4-yl |
| A-768. | OCH₃ | 5-methyl-6-trifluoromethylpyrimidin-4-yl |
| A-769. | OCH₃ | 6-trifluoromethylpyrimidin-4-yl |
| A-770. | OCH₃ | 2-methyl-5-fluoropyrimidin-4-yl |
| A-771. | OCH₃ | 2-methyl-5-chloropyrimidin-4-yl |
| A-772. | OCH₃ | 5-chloro-6-methylpyrimidin-4-yl |
| A-773. | OCH₃ | 5-chloro-6-ethylpyrimidin-4-yl |
| A-774. | OCH₃ | 5-chloro-6-isopropylpyrimidin-4-yl |
| A-775. | OCH₃ | 5-bromo-6-methylpyrimidin-4-yl |
| A-776. | OCH₃ | 5-fluoro-6-methylpyrimidin-4-yl |
| A-777. | OCH₃ | 5-fluoro-6-fluoromethylpyrimidin-4-yl |
| A-778. | OCH₃ | 2,6-dimethyl-5-chloropyrimidin-4-yl |
| A-779. | OCH₃ | 5,6-dimethylpyrimidin-4-yl |
| A-780. | OCH₃ | 2,5-dimethylpyrimidin-4-yl |
| A-781. | OCH₃ | 2,5,6-trimethylpyrimidin-4-yl |
| A-782. | OCH₃ | 5-methyl-6-methoxypyrimidin-4-yl |
| A-783. | OCH₃ | 5-pyrimidinyl |
| A-784. | OCH₃ | 4-methylpyrimidin-5-yl |
| A-785. | OCH₃ | 4,6-dimethylpyrimidin-5-yl |
| A-786. | OCH₃ | 2,4,6-trimethylpyrimidin-5-yl |
| A-787. | OCH₃ | 4-trifluoromethyl-6-methylpyrimidin-5-yl |
| A-788. | OCH₃ | 2-pyrimidinyl |
| A-789. | OCH₃ | 4,6-dimethylpyrimidin-2-yl |
| A-790. | OCH₃ | 4,5,6-trimethylpyrimidin-2-yl |
| A-791. | OCH₃ | 4,6-ditrifluoromethylpyrimidin-2-yl |
| A-792. | OCH₃ | 4,6-dimethyl-5-chloropyrimidin-2-yl |
| A-793. | Cl | phenyl |
| A-794. | Cl | 2-fluorophenyl |
| A-795. | Cl | 3-fluorophenyl |
| A-796. | Cl | 4-fluorophenyl |
| A-797. | Cl | 2-chlorophenyl |
| A-798. | Cl | 3-chlorophenyl |
| A-799. | Cl | 4-chlorophenyl |
| A-800. | Cl | 3-bromophenyl |
| A-801. | Cl | 4-bromophenyl |
| A-802. | Cl | 2-trifluoromethylphenyl |
| A-803. | Cl | 3-trifluoromethylphenyl |
| A-804. | Cl | 4-trifluoromethylphenyl |
| A-805. | Cl | 2-(methylthio)phenyl |
| A-806. | Cl | 3-(methylthio)phenyl |
| A-807. | Cl | 4-(methylthio)phenyl |
| A-808. | Cl | 2-methoxyphenyl |
| A-809. | Cl | 3-methoxyphenyl |
| A-810. | Cl | 4-methoxyphenyl |
| A-811. | Cl | 4-nitrophenyl |
| A-812. | Cl | 4-cyanophenyl |
| A-813. | Cl | 4-tert-butylphenyl |
| A-814. | Cl | 4-isopropylphenyl |
| A-815. | Cl | 3-ethoxyphenyl |
| A-816. | Cl | 4-ethoxyphenyl |
| A-817. | Cl | 4-n-propoxyphenyl |
| A-818. | Cl | 3-isopropoxyphenyl |
| A-819. | Cl | 4-isopropoxyphenyl |
| A-820. | Cl | 4-n-butoxyphenyl |
| A-821. | Cl | 4-tert-butoxyphenyl |
| A-822. | Cl | 4-(methoxyiminomethyl)phenyl |
| A-823. | Cl | 4-(1-(methoxyimino)ethyl)phenyl |
| A-824. | Cl | 2,3-difluorophenyl |
| A-825. | Cl | 2,4-difluorophenyl |
| A-826. | Cl | 2,5-difluorophenyl |
| A-827. | Cl | 3,4-difluorophenyl |
| A-828. | Cl | 3,5-difluorophenyl |
| A-829. | Cl | 2,6-difluorophenyl |
| A-830. | Cl | 2,4,6-trifluorophenyl |
| A-831. | Cl | 2,4,5-trifluorophenyl |
| A-832. | Cl | 2,3,4-trifluorophenyl |
| A-833. | Cl | 2,3,5-trifluorophenyl |
| A-834. | Cl | 3,4,5-trifluorophenyl |
| A-835. | Cl | 2,3-dichlorophenyl |
| A-836. | Cl | 2,5-dichlorophenyl |
| A-837. | Cl | 3,5-dichlorophenyl |
| A-838. | Cl | 2,6-dichlorophenyl |
| A-839. | Cl | 2,3-dimethylphenyl |
| A-840. | Cl | 2,4-dimethylphenyl |
| A-841. | Cl | 2,5-dimethylphenyl |
| A-842. | Cl | 2,4,5-trimethylphenyl |
| A-843. | Cl | 2,3-dimethoxyphenyl |
| A-844. | Cl | 2,4-dimethoxyphenyl |
| A-845. | Cl | 3,4-dimethoxyphenyl |
| A-846. | Cl | 2,4-bis(trifluoromethyl)phenyl |
| A-847. | Cl | 3,5-bis(trifluoromethyl)phenyl |
| A-848. | Cl | 2-methyl-3-methoxyphenyl |
| A-849. | Cl | 2-methyl-4-methoxyphenyl |
| A-850. | Cl | 2-methyl-6-methoxyphenyl |
| A-851. | Cl | 3-chloro-4-fluorophenyl |
| A-852. | Cl | 2-chloro-4-fluorophenyl |
| A-853. | Cl | 2-chloro-6-fluorophenyl |
| A-854. | Cl | 4-chloro-2-fluorophenyl |
| A-855. | Cl | 5-chloro-2-fluorophenyl |
| A-856. | Cl | 4-fluoro-3-methylphenyl |
| A-857. | Cl | 2-fluoro-4-methylphenyl |
| A-858. | Cl | 4-fluoro-2-methylphenyl |
| A-859. | Cl | 2-fluoro-3-methoxyphenyl |
| A-860. | Cl | 2-fluoro-4-methoxyphenyl |
| A-861. | Cl | 2-fluoro-6-methoxyphenyl |
| A-862. | Cl | 2-fluoro-4-trifluoromethylphenyl |
| A-863. | Cl | 4-chloro-3-methylphenyl |
| A-864. | Cl | 2-chloro-4-methylphenyl |
| A-865. | Cl | 2-chloro-6-methylphenyl |
| A-866. | Cl | 5-chloro-2-methylphenyl |
| A-867. | Cl | 3-chloro-2-methylphenyl |
| A-868. | Cl | 2-chloro-4-methoxyphenyl |
| A-869. | Cl | 2-chloro-6-methoxyphenyl |
| A-870. | Cl | 2-chloro-4-trifluoromethylphenyl |
| A-871. | Cl | 3-fluoro-4-methylphenyl |
| A-872. | Cl | 3-fluoro-4-isopropylphenyl |
| A-873. | Cl | 4-fluoro-3-methylphenyl |
| A-874. | Cl | 3-fluoro-4-methoxyphenyl |
| A-875. | Cl | 3-fluoro-4-ethoxyphenyl |
| A-876. | Cl | 3-fluoro-4-trifluoromethylphenyl |
| A-877. | Cl | 3-chloro-4-methylphenyl |
| A-878. | Cl | 3-chloro-4-methoxyphenyl |
| A-879. | Cl | 3-chloro-4-ethoxyphenyl |
| A-880. | Cl | 3-chloro-4-trifluoromethylphenyl |
| A-881. | Cl | 3-methyl-4-methoxyphenyl |
| A-882. | Cl | 4-chloro-2,5-difluorophenyl |
| A-883. | Cl | 4-tert-butyl-2-fluorophenyl |
| A-884. | Cl | 2-fluoro-4-isopropylphenyl |
| A-885. | Cl | 4-ethoxy-2-fluorophenyl |
| A-886. | Cl | 4-acetyl-2-fluorophenyl |
| A-887. | Cl | 2-thienyl |
| A-888. | Cl | 5-methylthiophen-2-yl |
| A-889. | Cl | 4-methylthiophen-2-yl |
| A-890. | Cl | 5-chlorothiophen-2-yl |
| A-891. | Cl | 3-cyanothiophen-2-yl |
| A-892. | Cl | 5-formylthiophen-2-yl |
| A-893. | Cl | 5-acetylthiophen-2-yl |
| A-894. | Cl | 5-(methoxyiminomethyl)thiophen-2-yl |
| A-895. | Cl | 5-(1-(methoxyimino)ethyl)thiophen-2-yl |
| A-896. | Cl | 4-bromothiophen-2-yl |
| A-897. | Cl | 3,5-dichlorothiophen-2-yl |
| A-898. | Cl | 3-thienyl |
| A-899. | Cl | 2-methylthiophen-3-yl |
| A-900. | Cl | 2,5-dichlorothiophen-3-yl |
| A-901. | Cl | 2,4,5-trichlorothiophen-3-yl |
| A-902. | Cl | 2-furyl |
| A-903. | Cl | 5-methylfuran-2-yl |
| A-904. | Cl | 5-chlorofuran-2-yl |
| A-905. | Cl | 4-methylfuran-2-yl |
| A-906. | Cl | 3-cyanofuran-2-yl |
| A-907. | Cl | 5-acetylfuran-2-yl |
| A-908. | Cl | 3-furyl |
| A-909. | Cl | 2-methylfuran-3-yl |
| A-910. | Cl | 2,5-dimethylfuran-3-yl |
| A-911. | Cl | 2-pyridyl |
| A-912. | Cl | 3-fluoropyridin-2-yl |
| A-913. | Cl | 3-chloropyridin-2-yl |
| A-914. | Cl | 3-bromopyridin-2-yl |
| A-915. | Cl | 3-trifluoromethylpyridin-2-yl |
| A-916. | Cl | 3-methylpyridin-2-yl |
| A-917. | Cl | 3-ethylpyridin-2-yl |
| A-918. | Cl | 3,5-difluoropyridin-2-yl |
| A-919. | Cl | 3,5-dichloropyridin-2-yl |
| A-920. | Cl | 3,5-dibromopyridin-2-yl |
| A-921. | Cl | 3,5-dimethylpyridin-2-yl |
| A-922. | Cl | 3-fluoro-5-trifluoromethylpyridin-2-yl |
| A-923. | Cl | 3-chloro-5-fluoropyridin-2-yl |
| A-924. | Cl | 3-chloro-5-methylpyridin-2-yl |
| A-925. | Cl | 3-fluoro-5-chloropyridin-2-yl |
| A-926. | Cl | 3-fluoro-5-methylpyridin-2-yl |
| A-927. | Cl | 3-methyl-5-fluoropyridin-2-yl |
| A-928. | Cl | 3-methyl-5-chloropyridin-2-yl |
| A-929. | Cl | 5-nitropyridin-2-yl |
| A-930. | Cl | 5-cyanopyridin-2-yl |
| A-931. | Cl | 5-methoxycarbonylpyridin-2-yl |
| A-932. | Cl | 5-trifluoromethylpyridin-2-yl |
| A-933. | Cl | 5-methylpyridin-2-yl |
| A-934. | Cl | 4-methylpyridin-2-yl |
| A-935. | Cl | 6-methylpyridin-2-yl |
| A-936. | Cl | 3-pyridyl |
| A-937. | Cl | 2-chloropyridin-3-yl |
| A-938. | Cl | 2-bromopyridin-3-yl |
| A-939. | Cl | 2-methylpyridin-3-yl |
| A-940. | Cl | 2,4-dichloropyridin-3-yl |
| A-941. | Cl | 2,4-dibromopyridin-3-yl |
| A-942. | Cl | 2,4-difluoropyridin-3-yl |
| A-943. | Cl | 2-fluoro-4-chloropyridin-3-yl |
| A-944. | Cl | 2-chloro-4-fluoropyridin-3-yl |
| A-945. | Cl | 2-chloro-4-methylpyridin-3-yl |
| A-946. | Cl | 2-methyl-4-fluoropyridin-3-yl |
| A-947. | Cl | 2-methyl-4-chloropyridin-3-yl |
| A-948. | Cl | 2,4-dimethylpyridin-3-yl |
| A-949. | Cl | 2,4,6-trichloropyridin-3-yl |
| A-950. | Cl | 2,4,6-tribromopyridin-3-yl |
| A-951. | Cl | 2,4,6-trimethylpyridin-3-yl |
| A-952. | Cl | 2,4-dichloro-6-methylpyridin-3-yl |
| A-953. | Cl | 4-pyridyl |
| A-954. | Cl | 3-chloropyridin-4-yl |
| A-955. | Cl | 3-bromopyridin-4-yl |
| A-956. | Cl | 3-methylpyridin-4-yl |
| A-957. | Cl | 3,5-dichloropyridin-4-yl |
| A-958. | Cl | 3,5-dibromopyridin-4-yl |
| A-959. | Cl | 3,5-dimethylpyridin-4-yl |
| A-960. | Cl | 4-pyrimidinyl |
| A-961. | Cl | 5-chloropyrimidin-4-yl |
| A-962. | Cl | 5-fluoropyrimidin-4-yl |
| A-963. | Cl | 5-fluoro-6-chloropyrimidin-4-yl |
| A-964. | Cl | 2-methyl-6-trifluoromethylpyrimidin-4-yl |
| A-965. | Cl | 2,5-dimethyl-6-trifluoromethylpyrimidin-4-yl |
| A-966. | Cl | 5-methyl-6-trifluoromethylpyrimidin-4-yl |
| A-967. | Cl | 6-trifluoromethylpyrimidin-4-yl |
| A-968. | Cl | 2-methyl-5-fluoropyrimidin-4-yl |
| A-969. | Cl | 2-methyl-5-chloropyrimidin-4-yl |
| A-970. | Cl | 5-chloro-6-methylpyrimidin-4-yl |
| A-971. | Cl | 5-chloro-6-ethylpyrimidin-4-yl |
| A-972. | Cl | 5-chloro-6-isopropylpyrimidin-4-yl |
| A-973. | Cl | 5-bromo-6-methylpyrimidin-4-yl |
| A-974. | Cl | 5-fluoro-6-methylpyrimidin-4-yl |
| A-975. | Cl | 5-fluoro-6-fluoromethylpyrimidin-4-yl |
| A-976. | Cl | 2,6-dimethyl-5-chloropyrimidin-4-yl |
| A-977. | Cl | 5,6-dimethylpyrimidin-4-yl |
| A-978. | Cl | 2,5-dimethylpyrimidin-4-yl |
| A-979. | Cl | 2,5,6-trimethylpyrimidin-4-yl |
| A-980. | Cl | 5-methyl-6-methoxypyrimidin-4-yl |
| A-981. | Cl | 5-pyrimidinyl |
| A-982. | Cl | 4-methylpyrimidin-5-yl |
| A-983. | Cl | 4,6-dimethylpyrimidin-5-yl |
| A-984. | Cl | 2,4,6-trimethylpyrimidin-5-yl |
| A-985. | Cl | 4-trifluoromethyl-6-methylpyrimidin-5-yl |
| A-986. | Cl | 2-pyrimidinyl |
| A-987. | Cl | 4,6-dimethylpyrimidin-2-yl |
| A-988. | Cl | 4,5,6-trimethylpyrimidin-2-yl |
| A-989. | Cl | 4,6-ditrifluoromethylpyrimidin-2-yl |
| A-990. | Cl | 4,6-dimethyl-5-chloropyrimidin-2-yl |

The compounds according to the invention of the general formula I can be prepared analogously to the prior art cited at the outset using standard methods of organic synthesis.

Compounds of the formula I in which R⁵ is optionally substituted phenyl or heteroaryl can be prepared, for example, according to the process shown in Scheme 1:

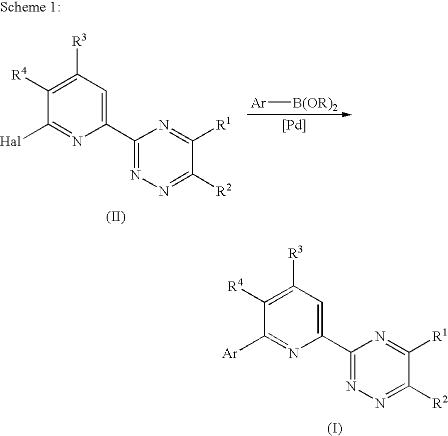

In Scheme 1, R¹, R², R³ and R⁴ are as defined above. Ar is optionally substituted phenyl or optionally substituted 5- or 6-membered hetaryl. R is H or C₁-C₄-alkyl or, together with further molecules Ar—B(OR)₂, forms a phenylboronic anhydride. Hal is chlorine, bromine or iodine.

According to Scheme 1, the 2-(6-halopyridin-2-yl)triazine of the formula II is reacted with a (het)arylboronic acid derivative of the general formula Ar—B(OR)₂ under the conditions of a Suzuki coupling, i.e. in the presence of a palladium catalyst under reaction conditions known per se as disclosed, for example, in Acc. Chem. Res. 15, pp. 178-184 (1982), Chem. Rev. 95, pp. 2457-2483 (1995), and the literature cited therein, and also in J. Org. Chem. 68, p. 9412 (2003). Suitable catalysts are in particular tetrakis(triphenylphosphine)palladium(0), bis(triphenylphosphine)palladium(II) chloride, bis(acetonitrile)palladium(II) chloride, the [1,1′-bis(diphenylphosphino)ferrocene]-palladium(II) chloride/dichloromethane complex, bis[1,2-bis(diphenylphosphine)ethane]palladium(0) and [1,4-bis(diphenylphosphine)butane]palladium(II) chloride. The amount of catalyst is usually form 0.1 to 10 mol %, based on the compound II. The molar ratio of compound II to the (het)arylboronic acid derivative is typically in the range from 1:2 to 2:1.

In a manner analogous to Scheme 1, compounds of the formula I in which R⁵ is alkyl, cycloalkyl, optionally substituted benzyl or heteroarylmethyl can be prepared via a transition metal-catalyzed coupling reaction by reacting compound II with an organometallic compound Met-R⁵ in which R⁵ is as defined above and Met is a radical MgX, SnR₃ or ZnX (X=chlorine, bromine or iodine, R=alkyl), for example in the sense of a Stille coupling or Kumada coupling.

In a manner analogous to Scheme 1, compounds of the formula I in which R⁵ is alkoxy, haloalkoxy, cycloalkoxy, optionally substituted phenoxy or heteroaryloxy can be prepared by reacting compound II with the appropriate alkoxide OR⁵ in which R⁵ is as defined above, in the sense of a nucleophilic substitution.

For their part, the 3-(6-halopyridin-2-yl)triazines of the formula II can be prepared by the methods shown in the schemes below from the corresponding amidine compounds of the formula Ill.

The preparation of compounds II in which R¹, R² independently of one another are alkyl, haloalkyl or together with the carbon atoms to which they are attached form a saturated carbocycle or a heterocycle according to the above definition can be achieved, for example, according to the synthesis shown in Scheme 2.

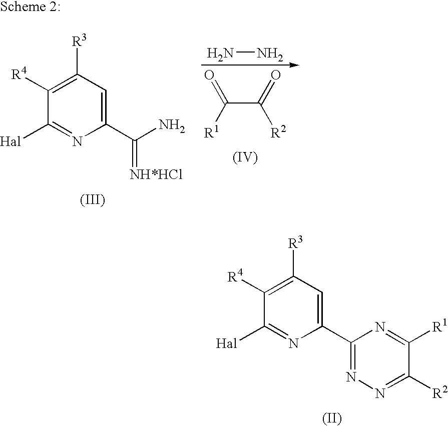

In Scheme 2, Hal, R³ and R⁴ are as defined above. R¹ and R² independently of one another are C₁-C₈-alkyl, C₁-C₈-haloalkyl or together with the carbon atoms to which they are attached form a saturated carbocycle or a heterocycle, for example one of the cycles Q-1, Q-4 or Q-8 mentioned above. According to Scheme 2, the amidine compound of the formula III is, successively or in a one-pot reaction, reacted with hydrazine and the compound IV, giving the compound of the formula II. In general, the reaction with the hydrazine, which is typically employed as hydrazine hydrate, is carried out first. This is followed by the addition of compound IV. If appropriate, the temperature is increased to bring the reaction to completion.

The compounds of the formula IV are known or can be prepared by standard processes, for example according to EP 267 378, Tetrahedron Letters, 2003, p. 2307, Tetrahedron Letters, 1992, p. 8131, Tetrahedron Letters, 1987, p. 551, J. Molec. Catalysis 208 (1-2), 2004, pp. 135-145. 3,4-Dioxaoxolane can be prepared, for example, by oxidizing the 3,4-bishydroxyoxolane with N-bromosuccinimide and carbon tetrachloride in pyridine (see Tetrahedron Letters, 44 (2003) p. 4909) or with o-iodosobenzoic acid in dimethyl sulfoxide (see Tetrahedron Letters, 35 (1994) S. 8019). The amidine compounds III are likewise known or can be prepared, for example, analogously to the methods described in US 2003/0087940 A1 and Bioorg. Med. Chem. Lett. 1571-1574 (2003).

In a manner analogously to Scheme II, the compounds of the formula II in which R¹ and R² together with the carbon atoms to which they are attached are a radical Q-2 or Q-3 can be prepared by the synthesis route shown in Schemes 3 and 4:

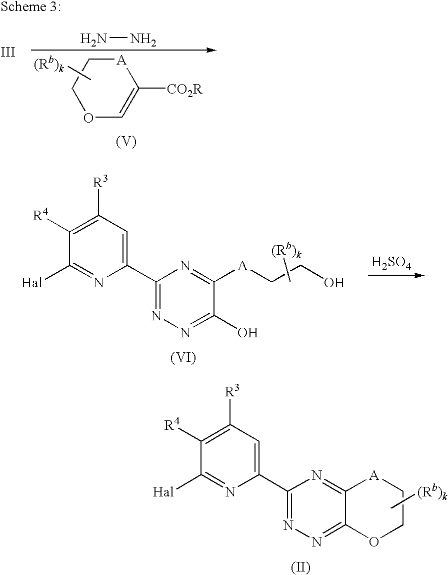

In Scheme 3, Hal, k, R^{b}, R³ and R⁴ are as defined above. A is CH₂ or a chemical bond. R is C₁-C₄-alkyl, in particular methyl or ethyl. According to scheme 3, the amidine compound III is reacted successively with hydrazine and the ester of the formula V. With respect to the reaction conditions, what was stated above for Scheme 2 applies analogously. The bishydroxy compound of the formula VI obtained in this manner is then subjected to a cyclizing dehydration, for example by treatment with sulfuric acid. The esters of the formula V are known or can be prepared analogously to processes known from the literature (see J. Heterocycl. Chem., 32 (1995) p. 735 and Liebigs Ann. Chem. 1974, pp. 468-476).

For their part, the compounds of the general formula III can be prepared from the corresponding 2-cyanopyridine compounds of the general formula VII (see Scheme 4). To this end, the 2-cyanopyridine compound VII is, using the method described in U.S. Pat. No. 4,873,248, converted by successive treatment with alkali metal alkoxide, such as sodium methoxide or ethoxide, and subsequent reaction with ammonium chloride, into the compound II. Instead of the hydrochlorides, it is also possible to use the hydrobromides, acetates, sulfates or formates in the subsequent steps shown in Schemes 1 to 3. The cyanopyridines of the formula VII are known, for example from US 2003/087940, WO 2004/026305, WO 01/057046 and Bioorg. Med. Chem. Lett. pp. 1571-1574 (2003), or they can be prepared by known preparation processes.

According to a second synthesis route (see Scheme 4), the compounds according to the invention in which R⁵ is a radical Ar as defined in Scheme 1 can be prepared from the cyanopyridines VII. To this end, the compound VII is initially coupled with the (het)arylboronic acid compound Ar—B(OR)₂, as described for Scheme 1, and the resulting 6-(het)aryl-2-cyanopyridine is converted under the reaction conditions described for compounds VII into the amidine compound IX. Compound IX can then be converted under the conditions mentioned for Schemes 2 and 3 into the corresponding triazine compound.

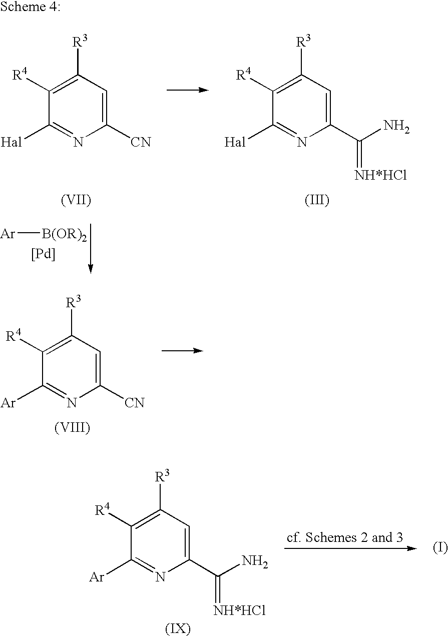

Besides, compounds of the formula VIIIa in which R⁵ has one of the meanings mentioned above, in particular a meaning different from Ar, are known. Using methods analogous to Schemes 2 to 4, the compounds VIIIa can be converted into the corresponding compounds I according to the invention:

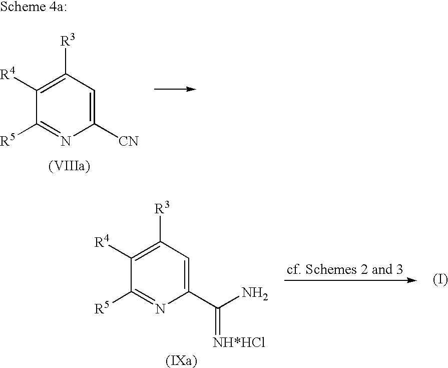

Compounds of the general formula VII can, if they are not already known, be prepared in particular by the process shown in Scheme 5.

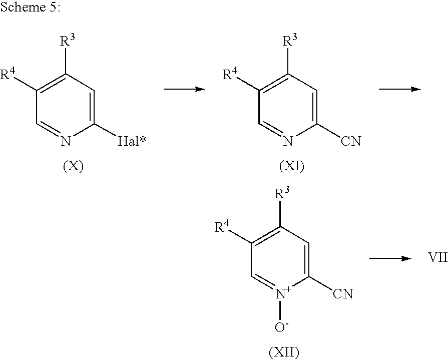

In Scheme 5, R³ and R⁴ are as defined above. Hal* is chlorine, bromine or iodine.

The conversion of the 2-halopyridine X into the 2-cyanopyridine XI is performed using standard methods of organic chemistry by reacting X with cyanide ions, for example with sodium or potassium cyanide (see EP-A 97460, preparation example 1), copper(I) cyanide (see EP-A 34917, preparation example 3) or trimethylsilyl cyanide. The compound XI obtained in this manner is then converted by treatment with a peracid using methods known per se into the pyridine N-oxide XII. The conversion of XI into XII may be carried out analogously to known processes, for example by treating XI with hydrogen peroxide in an organic acid such as formic acid, acetic acid, chloroacetic acid or trifluoroacetic acid (see, for example, J. Org. Chem. 55, pp. 738-741 (1990) and Organic Synthesis, Collect. Vol. IV, pp. 655-656 (1963)) or by reacting XI with an organic peracid, such as meta-chloroperbenzoic acid, in an inert solvent, for example a halogenated hydrocarbon, such as dichloromethane or dichloroethane (see, for example, Synthetic Commun. 22(18), p. 2645, (1992); J. Med. Chem. 2146 (1998)). The conversion of XI into XII can also be carried out analogously to the method described by K. B. Sharpless (J. Org. Chem. 63(5), p. 7740 (1998)) by reacting XI in a halogenated hydrocarbon, such as dichloromethane or dichloroethane, in the presence of catalytic amounts (for example 5% by weight) of rhenium(VII) compounds, such as methyltrioxorhenium (H₃CReO₃), with hydrogen peroxide.

XII is then reacted with a halogenating agent, such as POCl₃ or POBr₃, which yields the corresponding compound VII. In the case of the conversion of XII into VII the halogenating agent is generally employed in excess, based on the stoichiometry of the reaction. The reaction can be carried out in an inert organic solvent and is frequently carried out in the absence of a solvent, the halogenating agent then generally acting as solvent. The reaction temperature is usually in the range of from 20° C. to the boiling point of the halogenating agent. If appropriate, it is advantageous initially to introduce a chlorine atom into the 2-position of the pyridine N-oxide using a chlorinating agent such as POCl₃ and then to carry out a halogen exchange, for example by treatment with HBr or an iodinating agent, giving a compound of the formula VII where Hal=Br or I.

The compounds I according to the invention can furthermore be prepared according to the synthesis shown in Scheme 6:

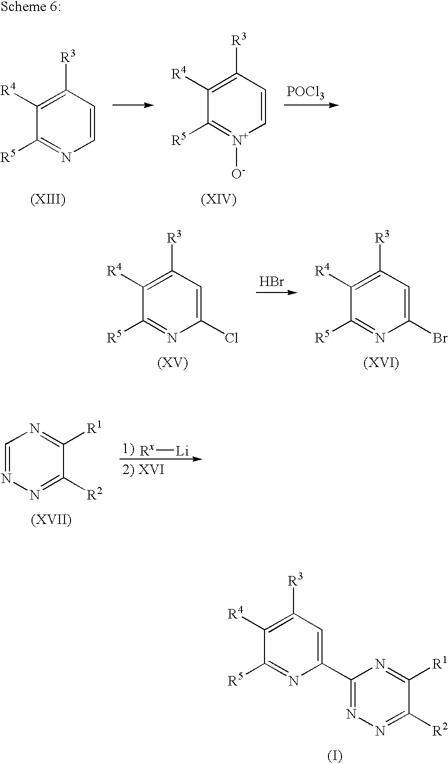

In Scheme 6, R¹, R², R³, R⁴ and R⁵ are as defined above. R^{x} is, for example, C₁-C₆-alkyl, especially n-butyl, or phenyl.

According to Scheme 6, in a first step the pyridine compound XIII is, according to standard methods of organic chemistry, converted into the corresponding N-oxide XIV. The methods mentioned in Scheme 5 for the conversion of the compound XI into the compound XII can be applied correspondingly. By reaction with a chlorinating agent, such as POCl₃, the N-oxide XIV is then converted into the corresponding 2-chloropyridine XV. The methods shown in Scheme 5 for the conversion of the compound XII into the compound VII can be applied correspondingly. This is then followed by a chlorine-bromine exchange according to standard methods of organic chemistry, for example by treating the chloropyridine XV with HBr analogously to the method mentioned in U.S. Pat. No. 5,271,217 and Can. J. Chem. 75 (2) (1997) p. 169.

The bromopyridine obtained in this manner is then coupled with the triazine compound XVII. To this end, the triazine compound of the formula XVII is, by treatment with a lithium base, in particular a C₁-C₆-alkyllithium compound, such as n-butyllithium or phenyllithium, converted into the corresponding lithiumtriazine compound, which is then reacted with the bromopyridine compound XVI. The reaction of XVI with the lithiated compound XVII can be carried out analogously to the method described in Tetrahedron Lett. 41(10) (2000) p. 1653.

The triazine compounds XVII are known or can be prepared analogously to known methods for preparing triazine compounds. Triazine compounds XVII in which R¹ and R² are radicals attached via oxygen can be prepared from 5,6-dialkoxy-1,2,4-triazines, such as 5,6-dimethoxy-1,2,4-triazine (see Chem. Ber. 109 (1976), p. 1113):

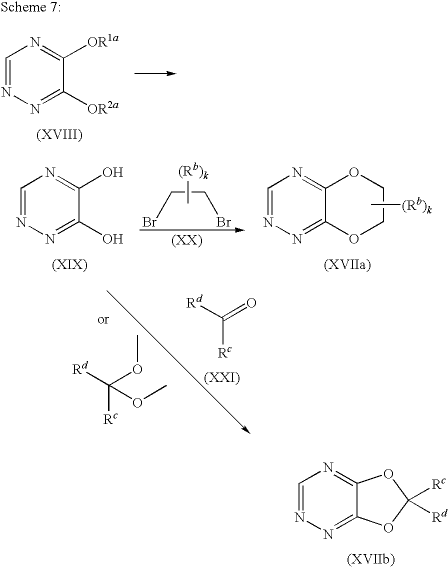

In Schema 7, R^{b} and k are as defined above. R^{1a} and R^{1b} are C₁-C₆-alkyl, in particular methyl. R^{c} and R^{d} independently of one another are hydrogen or C₁-C₄-alkyl. The conversion of XVIII into the 5,6-bishydroxy-1,2,4-triazine can be performed analogously to the method described in Chem. Ber. 109 (1976), p. 1113. The bishydroxytriazine XIX obtained in this manner can then be reacted with an 1,2-dibromoalkane XX, preferably in the presence of a base, such as an alkali metal hydroxide or alkali metal alkoxide, analogously to the method described in Heterocycl. Chem. 27 (1990) p., 151, which gives the condensed triazine XVIIa. Moreover, the bishydroxytriazine XIX can, analogously to the method described in Chem. Berichte 124(3) (1991) p. 481, J. Chem. Socl., Perkin Trans 1, 1998, p. 3561; Synthesis (1986), p. 122, be reacted with a ketone or an aldehyde XXI, which gives the condensed triazine XVIIb.

The reaction mixtures obtained by the processes shown in Schemes 1 to 7 are worked up in a customary manner, for example by mixing with water, separating the phases and, if appropriate, chromatographic purification of the crude products. Some of the intermediates and end products are obtained in the form of colorless or slightly brownish viscous oils which are purified or freed from volatile components under reduced pressure and at moderately elevated temperature. If the intermediates and end products are obtained as solids, purification can also be carried out by recrystallization or digestion.

If individual compounds I cannot be obtained by the routes described above, they can be prepared by derivatization of other compounds I.

If the synthesis yields mixtures of isomers, a separation is generally however not necessarily required since in some cases the individual isomers can be interconverted during work-up for use or during application (for example under the action of light, acids or bases). Such conversions may also take place after use, for example, in the case of treatment of plants, in the treated plant, or in the harmful fungus to be controlled.

The compounds of the formula I are suitable as fungicides. They are distinguished by excellent activity against a broad spectrum of phytopathogenic fungi from the classes of the Ascomycetes, Deuteromycetes, Oomycetes and Basidiomycetes, in particular from the class of the Oomycetes. Some of them are systemically active and can be used in crop protection as foliar fungicides, as fungicides for seed dressing and as soil fungicides.

They are particularly important in the control of a large number of fungi on various crop plants, such as wheat, rye, barley, oats, rice, corn, grass, bananas, cotton, soybeans, coffee, sugar cane, grapevines, fruit and ornamental plants and vegetable plants, such as cucumbers, beans, tomatoes, potatoes and cucurbits, and also on the seeds of these plants.

They are especially suitable for controlling the following plant diseases:
Alternaria species on vegetables, oilseed rape, sugar beet, fruit, rice, soybeans, and also on potatoes (for example A. solani or A. alternata) and tomatoes (for example A. solani or A. alternata) and Alternaria ssp. (black head) on wheat,
Aphanomyces species on sugar beet and vegetables,
Ascochyta species on cereals and vegetables, for example Ascochyta tritici (leaf spot) on wheat,
Bipolaris and Drechslera species on corn, cereals, rice and lawn (for example D. maydis on corn, D. teres on barley, D. tritici-repentis on wheat),
Blumeria graminis (powdery mildew) on cereals (for example wheat or barley),
Botrytis cinerea (gray mold) on strawberries, vegetables, flowers, grapevines and wheat,
Bremia lactucae on lettuce,
Cercospora species on corn, soybeans, rice and sugar beet and, for example, Cercospora sojina (leaf blotch) or Cercospora kikuchii (leaf blotch) on soybeans,
Cladosporium herbarum (black mold) on wheat,
Cochliobolus species on corn, cereals and rice (for example Cochliobolus sativus on cereals and Cochliobolus miyabeanus on rice),
Colletotricum species on soybeans, cotton and other plants (for example C. acutatum on various plants and, for example, Colletotrichum truncatum (antracnose) on soybeans),
Corynespora cassiicola (leaf blotch) on soybeans,
Dematophora necatrix (root/stem rot) on soybeans,
Diaporthe phaseolorum (stem disease) on soybeans,
Drechslera species, Pyrenophora species on corn, cereals, rice and lawn, on barley (for example D. teres) and on wheat (for example D. tritici-repentis),
Esca on grapevines, caused by Phaeoacremonium chlamydosporium, Ph. Aleophilum, and Formitipora punctata (syn. Phellinus punctatus),
Elsinoe ampelina on grapevines,
Epicoccum spp. (black head) on wheat,
Exserohilum species on corn,
Erysiphe cichoracearum and Sphaerotheca fuliginea on cucumbers,
Fusarium and Verticillium species (for example V. dahliae) on various plants: for example F. graminearum or F. culmorum (root rot) on cereals (for example wheat or barley) or, for example, F. oxysporum on tomatoes and Fusarium solani (stem disease) on soybeans,
Gaeumanomyces graminis on cereals (for example wheat or barley),
Gibberella species on cereals and rice (for example Gibberella fujikuroi on rice),
Glomerella cingulata on grapevines and other plants,
Grainstaining complex on rice,
Guignardia budwelli on grapevines,
Helminthosporium species (for example H. graminicola) on corn and rice,
Isariopsis clavispora on grapevines,
Macrophomina phaseolina (root/stem rot) on soybeans,
Michrodochium nivale on cereals (for example wheat or barley),
Microsphaera diffusa (powdery mildew) on soybeans,
Mycosphaerella species on cereals, bananas and peanuts (M. graminicola on wheat, M. fijiensis on bananas),
Peronospora species on cabbage (for example P. brassicae), bulbous plants (for
example P. destructor) and, for example, Peronospora manshurica (downy mil dew) on soybeans,
Phakopsara pachyrhizi and Phakopsara meibomiae on soybeans,
Phialophora gregata (stem disease) on soybeans,
Phomopsis species on soybeans, sunflowers and grapevines (P. viticola on grapevines, P. helianthii on sunflowers),
Phytophthora species on various plants, for example P. capsici on bell peppers, Phytophthora megasperma (leaf/stem rot) on soybeans, Phytophthora infestans on potatoes and tomatoes,
Plasmopara viticola on grapevines,
Podosphaera leucotricha on apples,
Pseudocercosporella herpotrichoides on cereals,
Pseudoperonospora species on hops and cucumbers (for example P. cubensis on cucumbers or P. humili on hops),
Pseudopezicula tracheiphilai on grapevines,
Puccinia species on various plants, for example P. triticina, P. striformins, P. hordei or P. graminis on cereals (for example wheat or barley) or on asparagus (for example P. asparagi),
Pyrenophora species on cereals,
Pyricularia oryzae, Corticium sasakii, Sarocladium oryzae, S. attenuatum, Entyloma oryzae on rice,
Pyricularia grisea on lawn and cereals,
Pythium spp. on lawn, rice, corn, cotton, oilseed rape, sunflowers, sugar beet, vegetables and other plants (for example P. ultimum or P. aphanidermatum),
Ramularia collo-cygni (physiological leaf spots) on barley,
Rhizoctonia species (for example R. solani) on cotton, rice, potatoes, lawn, corn, oilseed rape, sugar beet, vegetables and on other plants, for example Rhizoctonia solani (root/stem rot) on soybeans or Rhizoctonia cerealis (yellow patch) on wheat or barley,
Rhynchosporium secalis on barley (scald), rye and triticale,
Sclerotinia species on oilseed rape, sunflowers and other plants, for example, Sclerotinia sclerotiorum (stem disease) or Sclerotinia rolfsii (stem disease) on soybeans,
Septoria glycines (brown spot) on soybeans,
Septoria tritici and Stagonospora nodorum on wheat,
Erysiphe (syn. Uncinula necator) on grapevines,
Setospaeria species on corn and lawn,
Sphacelotheca reilinia on corn,
Stagonospora nodorum (glume blotch) on wheat,
Thievaliopsis species on soybeans and cotton,
Tilletia species on cereals,
Typhula incarnata (snow mold) on wheat or barley,
Ustilago species on cereals, corn and sugar beet,
Venturia species (scab) on apples and pears (for example V. inaequalis on apples).

The compounds of the formula I are furthermore suitable for controlling harmful fungi in the protection of materials (for example wood, paper, paint dispersions, fibers or fabrics) and in the protection of stored products. In the protection of wood, particular attention is paid to the following harmful fungi:

Ascomycetes, such as Ophiostoma spp., Ceratocystis spp., Aureobasidium pullulans, Sclerophoma spp., Chaetomium spp., Humicola spp., Petriella spp., Trichurus spp.; Basidiomycetes, such as Coniophora spp., Coriolus spp., Gloeophyllum spp., Lentinus spp., Pleurotus spp., Poria spp., Serpula spp. and Tyromyces spp., Deuteromycetes, such as Aspergillus spp., Cladosporium spp., Penicillium spp., Trichoderma spp., Alternaria spp., Paecilomyces spp. and Zygomycetes, such as Mucor spp., additionally in the protection of materials the following yeasts: Candida spp. and Saccharomyces cerevisae.

The compounds of the formula I are employed by treating the fungi or the plants, seeds, materials or soil to be protected from fungal attack with a fungicidally effective amount of the active compounds. The application can be carried out both before and after the infection of the materials, plants or seeds by the fungi.

The fungicidal compositions generally comprise between 0.1 and 95%, preferably between 0.5 and 90%, by weight of active compound.

When employed in plant protection, the amounts applied are, depending on the kind of effect desired, between 0.01 and 2.0 kg of active compound per ha.

In seed treatment amounts of active compound of from 1 to 1000 g/100 kg, preferably from 5 to 100 g/100 kg, of seed are generally necessary.

When used in the protection of materials or stored products, the amount of active compound applied depends on the kind of application area and on the desired effect. Amounts customarily applied in the protection of materials are, for example, 0.001 g to 2 kg, preferably 0.005 g to 1 kg, of active compound per cubic meter of treated material.

The compounds of the formula I can be present in different crystal modifications which may differ in their biological activity. They also form part of the subject matter of the present invention.

The compounds of the formula I can be converted into the customary formulations, for example solutions, emulsions, suspensions, dusts, powders, pastes and granules. The use form depends on the particular intended purpose; in each case, it should ensure a fine and even distribution of the compound according to the invention.

The formulations are prepared in a known manner, for example by extending the active compound with solvents and/or carriers, if desired using emulsifiers and dispersants. Solvents/auxiliaries suitable for this purpose are essentially:
water, aromatic solvents (for example Solvesso products, xylene), paraffins (for example mineral oil fractions), alcohols (for example methanol, butanol, pentanol, benzyl alcohol), ketones (for example cyclohexanone, gamma-butyrolactone), pyrrolidones (NMP, NOP), acetates (glycol diacetate), glycols, fatty acid dimethylamides, fatty acids and fatty acid esters. In principle, solvent mixtures may also be used,
carriers such as ground natural minerals (for example kaolins, clays, talc, chalk) and ground synthetic minerals (for example highly disperse silica, silicates); emulsifiers such as nonionogenic and anionic emulsifiers (for example polyoxyethylene fatty alcohol ethers, alkylsulfonates and arylsulfonates) and dispersants such as lignosulfite waste liquors and methylcellulose.

Suitable surfactants used are alkali metal, alkaline earth metal and ammonium salts of lignosulfonic acid, naphthalenesulfonic acid, phenolsulfonic acid, dibutylnaphthalenesulfonic acid, alkylarylsulfonates, alkyl sulfates, alkylsulfonates, fatty alcohol sulfates, fatty acids and sulfated fatty alcohol glycol ethers, furthermore condensates of sulfonated naphthalene and naphthalene derivatives with formaldehyde, condensates of naphthalene or of naphthalenesulfonic acid with phenol and formaldehyde, polyoxyethylene octylphenyl ether, ethoxylated isooctylphenol, octylphenol, nonylphenol, alkylphenyl polyglycol ethers, tributylphenyl polyglycol ether, tristearylphenyl polyglycol ether, alkylaryl polyether alcohols, alcohol and fatty alcohol ethylene oxide condensates, ethoxylated castor oil, polyoxyethylene alkyl ethers, ethoxylated polyoxypropylene, lauryl alcohol polyglycol ether acetal, sorbitol esters, lignosulfite waste liquors and methylcellulose.

Substances which are suitable for the preparation of directly sprayable solutions, emulsions, pastes or oil dispersions are mineral oil fractions of medium to high boiling point, such as kerosene or diesel oil, furthermore coal tar oils and oils of vegetable or animal origin, aliphatic, cyclic and aromatic hydrocarbons, for example toluene, xylene, paraffin, tetrahydronaphthalene, alkylated naphthalenes or their derivatives, methanol, ethanol, propanol, butanol, cyclohexanol, cyclohexanone, isophorone, highly polar solvents, for example dimethyl sulfoxide, N-methylpyrrolidone and water.

Powders, materials for spreading and dustable products can be prepared by mixing or concomitantly grinding the active substances with a solid carrier.

Granules, for example coated granules, impregnated granules and homogeneous granules, can be prepared by binding the active compounds to solid carriers. Examples of solid carriers are mineral earths such as silica gels, silicates, talc, kaolin, attaclay, limestone, lime, chalk, bole, loess, clay, dolomite, diatomaceous earth, calcium sulfate, magnesium sulfate, magnesium oxide, ground synthetic materials, fertilizers, such as, for example, ammonium sulfate, ammonium phosphate, ammonium nitrate, ureas, and products of vegetable origin, such as cereal meal, tree bark meal, wood meal and nutshell meal, cellulose powders and other solid carriers.

In general, the formulations comprise from 0.01 to 95% by weight, preferably from 0.1 to 90% by weight, of the active compound. The active compounds are employed in a purity of from 90% to 100%, preferably 95% to 100% (according to NMR spectrum).

The following are examples of formulations:

1. Products for dilution with water

### A Water-Soluble Concentrates (SL, LS)

10 parts by weight of the active compounds are dissolved in 90 parts by weight of water or in a water-soluble solvent. As an alternative, wetters or other auxiliaries are added. The active compound dissolves upon dilution with water. In this way, a formulation having a content of 10% by weight of active compound is obtained.

### B Dispersible Concentrates (DC)

20 parts by weight of the active compounds are dissolved in 70 parts by weight of cyclohexanone with addition of 10 parts by weight of a dispersant, for example polyvinylpyrrolidone. Dilution with water gives a dispersion. The active compound content is 20% by weight

### C Emulsifiable Concentrates (EC)

15 parts by weight of the active compounds are dissolved in 75 parts by weight of xylene with addition of calcium dodecylbenzenesulfonate and castor oil ethoxylate (in each case 5 parts by weight). Dilution with water gives an emulsion. The formulation has an active compound content of 15% by weight.

### D Emulsions (EW, EO, ES)

25 parts by weight of the active compounds are dissolved in 35 parts by weight of xylene with addition of calcium dodecylbenzenesulfonate and castor oil ethoxylate (in each case 5 parts by weight). This mixture is introduced into 30 parts by weight of water by means of an emulsifying machine (e.g. Ultraturax) and made into a homogeneous emulsion. Dilution with water gives an emulsion. The formulation has an active compound content of 25% by weight.

### E Suspensions (SC, OD, FS)

In an agitated ball mill, 20 parts by weight of the active compounds are comminuted with addition of 10 parts by weight of dispersants and wetters and 70 parts by weight of water or an organic solvent to give a fine active compound suspension. Dilution with water gives a stable suspension of the active compound. The active compound content in the formulation is 20% by weight.

### F Water-Dispersible Granules and Water-Soluble Granules (WG, SG)

50 parts by weight of the active compounds are ground finely with addition of 50 parts by weight of dispersants and wetters and prepared as water-dispersible or water-soluble granules by means of technical appliances (for example extrusion, spray tower, fluidized bed). Dilution with water gives a stable dispersion or solution of the active compound. The formulation has an active compound content of 50% by weight.

### G Water-Dispersible Powders and Water-Soluble Powders (WP, SP, SS, WS)

75 parts by weight of the active compounds are ground in a rotor-stator mill with addition of 25 parts by weight of dispersants, wetters and silica gel. Dilution with water gives a stable dispersion or solution of the active compound. The active compound content of the formulation is 75% by weight.

### H Gel Formulations

In a ball mill, 20 parts by weight of the active compounds, 10 parts by weight of dispersant, 1 part by weight of gelling agent and 70 parts by weight of water or an organic solvent are ground to give a fine suspension. On dilution with water, a stable suspension having an active compound content of 20% by weight is obtained.

2. Products to be applied undiluted

### I Dustable Powders (DP, DS)

5 parts by weight of the active compounds are ground finely and mixed intimately with 95 parts by weight of finely divided kaolin. This gives a dustable product having an active compound content of 5% by weight.

### J Granules (GR, FG, GG, MG)

0.5 parts by weight of the active compounds is ground finely and associated with 99.5 parts by weight of carriers. Current methods are extrusion, spray-drying or the fluidized bed. This gives granules to be applied undiluted having an active compound content of 0.5% by weight.

### K ULV Solutions (UL)

10 parts by weight of the active compounds are dissolved in 90 parts by weight of an organic solvent, for example xylene. This gives a product to be applied undiluted having an active compound content of 10% by weight.

For seed treatment, use is usually made of water-soluble concentrates (LS), suspensions (FS), dustable powders (DS), water-dispersible and water-soluble powders (WS, SS), emulsions (ES), emulsifiable concentrates (EC) and gel formulations (GF). These formulations can be applied to the seed in undiluted form or, preferably, diluted. Application can be carried out prior to sowing.

The active compounds can be used as such, in the form of their formulations or the use forms prepared therefrom, for example in the form of directly sprayable solutions, powders, suspensions or dispersions, emulsions, oil dispersions, pastes, dustable products, materials for spreading, or granules, by means of spraying, atomizing, dusting, spreading or pouring. The use forms depend entirely on the intended purposes; they are intended to ensure in each case the finest possible distribution of the active compounds according to the invention.

Aqueous use forms can be prepared from emulsion concentrates, pastes or wettable powders (wettable powders, oil dispersions) by adding water. To prepare emulsions, pastes or oil dispersions, the substances, as such or dissolved in an oil or solvent, can be homogenized in water by means of a wetter, tackifier, dispersant or emulsifier. However, it is also possible to prepare concentrates composed of active substance, wetter, tackifier, dispersant or emulsifier and, if appropriate, solvent or oil, and such concentrates are suitable for dilution with water.

The concentrations of active compound in the ready-for-use preparations can be varied within relatively wide ranges. In general, they are between 0.0001 and 10%, preferably between 0.01 and 1%.

The active compounds can also be used with great success in the ultra-low volume (ULV) process, it being possible to apply formulations with more than 95% by weight of active compound or even to apply the active compound without additives.

Oils of various types, wetting agents, adjuvants, herbicides, fungicides, other pesticides, or bactericides may be added to the active compounds, even, if appropriate, not until immediately prior to use (tank mix). These agents may be admixed with the compositions according to the invention in a weight ratio of from 1:100 to 100:1, preferably from 1:10 to 10:1.

Suitable adjuvants in this sense are in particular: organically modified polysiloxanes, for example Break Thru S 240®; alcohol alkoxylates, for example Atplus 245®, Atplus MBA 1303®, Plurafac LF 300® and Lutensol ON 30®; EO/PO block polymers, for example Pluronic RPE 2035® and Genapol B®; alcohol ethoxylates, for example Lutensol XP 80®; and sodium dioctylsulfosuccinate, for example Leophen RA®.

The compositions according to the invention can, in the use form as fungicides, also be present together with other active compounds, for example with herbicides, insecticides, growth regulators, fungicides or also with fertilizers. By mixing the compounds I or the compositions comprising them in the use form as fungicides with other fungicides, in many cases a broadening of the spectrum of fungicidal activity is achieved.

The following list of fungicides, with which the compounds according to the invention can be used in conjunction, is intended to illustrate the possible combinations without being limited thereto:

### Strobilurins

azoxystrobin, dimoxystrobin, enestroburin, fluoxastrobin, kresoxim-methyl, metominostrobin, picoxystrobin, pyraclostrobin, trifloxystrobin, orysastrobin, methyl (2-chloro-5-[1-(3-methylbenzyloxyimino)ethyl]benzyl)carbamate, methyl (2-chloro-5-[1-(6-methylpyridin-2-ylmethoxyimino)ethyl]benzyl)carbamate, methyl 2-(ortho-(2,5-dimethylphenyloxymethylene)phenyl)-3-methoxyacrylate;

### Carboxamides

carboxanilides: benalaxyl, benodanil, boscalid, carboxin, mepronil, fenfuram, fenhexamid, flutolanil, furametpyr, metalaxyl, ofurace, oxadixyl, oxycarboxin, penthiopyrad, thifluzamide, tiadinil, N-(4′-bromobiphenyl-2-yl)-4-difluoromethyl-2-methylthiazole-5-carboxamide, N-(4′-trifluoromethylbiphenyl-2-yl)-4-difluoromethyl-2-methylthiazole-5-carboxamide, N-(4′-chloro-3′-fluorobiphenyl-2-yl)-4-difluoromethyl-2-methylthiazole-5-carboxamide, N-(3′,4′-dichloro-4-fluorobiphenyl-2-yl)-3-difluoromethyl-1-methylpyrazole-4-carboxamide, N-(2-cyanophenyl)-3,4-dichloroisothiazole-5-carboxamide;
carboxylic acid morpholides: dimethomorph, flumorph;
benzamides: flumetover, fluopicolide (picobenzamid), zoxamide;
other carboxamides: carpropamid, diclocymet, mandipropamid, N-(2-(4-[3-(4-chlorophenyl)prop-2-ynyloxy]-3-methoxyphenyl)ethyl)-2-methanesulfonylamino-3-methylbutyramide, N-(2-(4-[3-(4-chlorophenyl)prop-2-ynyloxy]-3-methoxyphenyl)ethyl)-2-ethanesulfonylamino-3-methylbutyramide;

### Azoles

triazoles: bitertanol, bromuconazole, cyproconazole, difenoconazole, diniconazole, enilconazole, epoxiconazole, fenbuconazole, flusilazole, fluquinconazole, flutriafol, hexaconazole, imibenconazole, ipconazole, metconazole, myclobutanil, penconazole, propiconazole, prothioconazole, simeconazole, tebuconazole, tetraconazole, triadimenol, triadimefon, triticonazole;
imidazoles: cyazofamid, imazalil, pefurazoate, prochloraz, triflumizole;
benzimidazoles: benomyl, carbendazim, fuberidazole, thiabendazole;
others: ethaboxam, etridiazole, hymexazole;

### Nitrogenous Heterocyclyl Compounds

pyridines: fluazinam, pyrifenox, 3-[5-(4-chlorophenyl)-2,3-dimethylisoxazolidin-3-yl]-pyridine;
pyrimidines: bupirimate, cyprodinil, ferimzone, fenarimol, mepanipyrim, nuarimol,
pyrimethanil;
piperazines: triforine;
pyrroles: fludioxonil, fenpiclonil;
morpholines: aldimorph, dodemorph, fenpropimorph, tridemorph;
dicarboximides: iprodione, procymidone, vinclozolin;
others: acibenzolar-5-methyl, anilazine, captan, captafol, dazomet, diclomezine, fenoxanil, folpet, fenpropidin, famoxadone, fenamidone, octhilinone, probenazole, proquinazid, pyroquilon, quinoxyfen, tricyclazole, 5-chloro-7-(4-methylpiperidin-1-yl)-6-(2,4,6-trifluorophenyl)-[1,2,4]triazolo[1,5-a]pyrimidine, 2-butoxy-6-iodo-3-propylchromen-4-one, N,N-dimethyl-3-(3-bromo-6-fluoro-2-methylindole-1-sulfonyl)-[1,2,4]triazole-1-sulfonamide;

### Carbamates and Dithiocarbamates

dithiocarbamates: ferbam, mancozeb, maneb, metiram, metam, propineb, thiram, zineb, ziram;
carbamates: diethofencarb, flubenthiavalicarb, iprovalicarb, propamocarb, methyl 3-(4-chlorophenyl)-3-(2-isopropoxycarbonylamino-3-methylbutyrylamino)propionate, 4-fluorophenyl N-(1-(1-(4-cyanophenyl)ethanesulfonyl)but-2-yl)carbamate;

### Other Fungicides

guanidines: dodine, iminoctadine, guazatine;
antibiotics: kasugamycin, polyoxins, streptomycin, validamycin A;
organometallic compounds: fentin salts;
sulfur-containing heterocyclyl compounds: isoprothiolane, dithianon;
organophosphorus compounds: edifenphos, fosetyl, fosetyl-aluminum, iprobenfos, pyrazophos, tolclofos-methyl, phosphorous acid and its salts;
organochlorine compounds: thiophanate-methyl, chlorothalonil, dichlofluanid, tolylfluanid, flusulfamide, phthalide, hexachlorobenzene, pencycuron, quintozene;
nitrophenyl derivatives: binapacryl, dinocap, dinobuton;
inorganic active compounds: Bordeaux mixture, copper acetate, copper hydroxide, copper oxychloride, basic copper sulfate, sulfur;
others: spiroxamine, cyflufenamid, cymoxanil, metrafenone.

### PREPARATION EXAMPLES

### Example 1

### 5,6-Diethyl-3-[6-(4-fluorophenyl)-5-methylpyridin-2-yl]-[1,2,4]-triazine

### 1) Preparation of 6-bromo-5-methylpyridine-2-carboxamidine hydrochloride

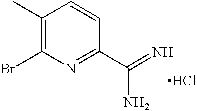

2.2 g of a 30% strength sodium methoxide solution in methanol were added to 4.90 g (25 mmol) of 6-bromo-5-methylpyridine-2-carbonitrile [preparation: see US 2003/0087940 A1 and Bioorg. Med. Chem. Lett. 1571-1574 (2003)] in 60 ml of methanol, and the mixture was stirred at 23° C. for 7 h. 1.5 g of ammonium chloride were then added, and the mixture was stirred at 23° C. for another 8 hours. After removal of the solvent, methyl tert-butyl ether (MtBE) was added and the product was filtered off with suction. Yield: 4.2 g as a white solid which was reacted further without purification.

### 2) Preparation of 3-(6-bromo-5-methylpyridin-2-yl)-5,6-diethyl-[1,2,4]-triazine

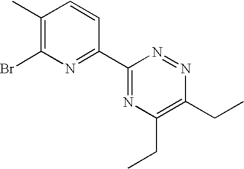

2.4 g (48 mmol) of hydrazinium hydroxide were added to a solution of 10 g (40 mmol) of the compound prepared in Step 1 in 100 ml of ethanol. After 30 min, 4.6 g (40 mmol) of 3,4-hexanedione were added and the mixture was stirred under reflux for 5 hours and then allowed to stand at 23° C. for 12 hours. Water and n-pentane were added to the reaction solution and the precipitated product was filtered off with suction. Yield: 9.9 g of product.

¹H-NMR (δ, CDCl₃,): 1.4 (m); 2.5 (s); 2.9 (m); 3.1 (m); 7.7 (m) and 8.4 (m).

### 3) Preparation of 5,6-diethyl-3-[6-(4-fluorophenyl)-5-methylpyridin-2-yl]-[1,2,4]-triazine

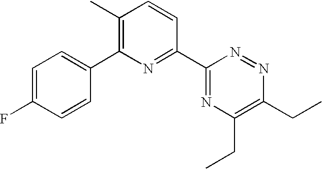

0.21 g of 4-fluorophenylboronic acid and 0.41 g of sodium carbonate in 20 ml of water were added successively to a solution of 0.4 g of the compound prepared in Step 2 in 20 ml of ethylene glycol dimethyl ether. After addition of about 30 mg of [1,4-bis(diphenylphosphino)butane]palladium(II) dichloride, the mixture was stirred under reflux for 5 hours. The reaction solution was then partitioned between water and MtBE. The organic phase was separated off, the solvent was removed under reduced pressure and the residue was chromatographed on silica gel using cyclohexane/MtBE (1:1). This gave 0.24 g of product.

M.p. 108° C.

### Example 2

### 3-[6-(4-Fluorophenyl)-5-methylpyridin-2-yl]-5,6,7,8-tetrahydrobenzo-[1,2,4]-triazine

### 1) Preparation of 3-(6-bromo-5-methylpyridin-2-yl)-5,6,7,8-tetrahydrobenzo-[1,2,4]-triazine

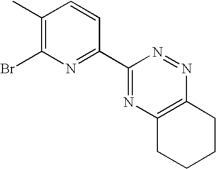

2.4 g (48 mmol) of hydrazinium hydroxide were added to a solution of 10 g (40 mmol) of 6-bromo-5-methylpyridine-2-carboxamidine hydrochloride in 100 ml of ethanol (EtOH). After 30 min, 4.5 g (40 mmol) of cyclohexane-1,2-dione were added, and the mixture was stirred under reflux for 7 hours. The reaction solution was then partitioned between water and MtBE, the organic phase was separated off, the solvent was removed under reduced pressure and the residue was chromatographed on silica gel using cyclohexane/MtBE (1:1) and MtBE/EtOH (1:1). Yield: 5.5 g of product.

¹H-NMR (6, CDCl₃,): 1.9 (m); 2.5 (s); 2.8 (m); 3.1 (m); 3.2 (m); 7.7 (m) and 8.45 (m).

### 2) Preparation of 3-[6-(4-fluorophenyl)-5-methylpyridin-2-yl]-5,6,7,8-tetrahydrobenzo-[1,2,4]-triazine

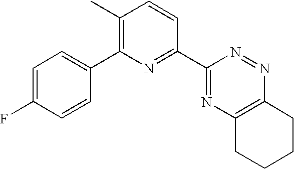

0.22 g of 4-fluorophenylboronic acid and 0.41 g of sodium carbonate in 20 ml of water were added successively to a solution of 0.4 g of the compound prepared in Step 1 in 20 ml of ethylene glycol dimethyl ether. After addition of about 30 mg of [1,4-bis(diphenylphosphino)butane]palladium(II) dichloride, the mixture was stirred under reflux for 5 hours. The reaction solution was then partitioned between water and MtBE. The organic phase was separated off, the solvent was removed under reduced pressure and the residue was chromatographed on silica gel using cyclohexane/MtBE (1:1). This gave 0.13 g of product.

M.p. 105° C.

The compounds of the general formula I described in Table 1 were prepared in an analogous manner:

TABLE 1

| | | | | | | Phys. data |
|---|---|---|---|---|---|---|
| No. | R⁵ | R⁴ | R³ | R¹ | R² | (° C.)/[M + H]⁺ |
| 1 | 4-fluorophenyl | CH₃ | H | CH₂CH₃ | CH₂CH₃ | 108 |
| 2 | 4-fluorophenyl | CH₃ | H | CH₂CH₂CH₂CH₂ | | 105 |
| 3 | 4-fluorophenyl | CH₃ | H | CH₂CH₃ | CH₂CH₃ | 112-115 |
| 4 | phenyl | CH₃ | H | CH₂CH₃ | CH₂CH₃ | 70-73 |
| 5 | 4-fluorophenyl | H | H | CH₃ | CH₃ | 151-154 |
| 6 | phenyl | CH₃ | H | CH₃ | CH₃ | 193-196 |
| 7 | 3,4-difluorophenyl | CH₃ | H | CH₃ | CH₃ | 139-142 |
| 8 | 3,4,5-trifluorophenyl | CH₃ | H | CH₃ | CH₃ | 169 |
| 9 | 3,5-difluorophenyl | CH₃ | H | CH₃ | CH₃ | 158 |
| 10 | 3-chloro-4-fluorophenyl | CH₃ | H | CH₃ | CH₃ | 120 |
| 11 | 3-methyl-4-fluorophenyl | CH₃ | H | CH₃ | CH₃ | 154 |
| 12 | 4-chlorophenyl | CH₃ | H | CH₂CH₃ | CH₂CH₃ | 133-137 |
| 13 | 4-methylphenyl | CH₃ | H | CH₂CH₃ | CH₂CH₃ | 128-130 |
| 14 | 4-methoxyphenyl | CH₃ | H | CH₂CH₃ | CH₂CH₃ | 118-120 |
| 15 | 4-cyanophenyl | CH₃ | H | CH₂CH₃ | CH₂CH₃ | 171-173 |
| 16 | 4-formylphenyl | CH₃ | H | CH₂CH₃ | CH₂CH₃ | 122-124 |
| 17 | 4-tert-butylphenyl | CH₃ | H | CH₂CH₃ | CH₂CH₃ | 97-101 |
| 18 | 4-isopropylphenyl | CH₃ | H | CH₂CH₃ | CH₂CH₃ | 100-103 |
| 19 | 4-trifluoromethylphenyl | CH₃ | H | CH₂CH₃ | CH₂CH₃ | 131-137 |
| 20 | 2-chlorophenyl | CH₃ | H | CH₂CH₃ | CH₂CH₃ | 103-106 |
| 21 | 2-fluorophenyl | CH₃ | H | CH₂CH₃ | CH₂CH₃ | 129-132 |
| 22 | 2-methylphenyl | CH₃ | H | CH₂CH₃ | CH₂CH₃ | 114-120 |
| 23 | 2,4-difluorophenyl | CH₃ | H | CH₂CH₃ | CH₂CH₃ | 142-144 |
| 24 | 3-chlorophenyl | CH₃ | H | CH₂CH₃ | CH₂CH₃ | 95-98 |
| 25 | 3-methylphenyl | CH₃ | H | CH₂CH₃ | CH₂CH₃ | 61-64 |
| 26 | 3-methoxyphenyl | CH₃ | H | CH₂CH₃ | CH₂CH₃ | oil |
| 27 | 4-acetylphenyl | CH₃ | H | CH₂CH₃ | CH₂CH₃ | 124-126 |
| 28 | 4-ethoxyphenyl | CH₃ | H | CH₂CH₃ | CH₂CH₃ | 103-107 |
| 29 | 3,4-dimethoxyphenyl | CH₃ | H | CH₂CH₃ | CH₂CH₃ | 99-102 |
| 30 | 3,5-dichlorophenyl | CH₃ | H | CH₂CH₃ | CH₂CH₃ | 146-148 |
| 31 | phenyl | CH₃ | H | CH₂CH₂CH₂CH₂ | | 161 |
| 32 | 4-fluorophenyl | H | H | CH₂CH₂CH₂CH₂ | | 140-143 |
| 33 | 3-chloro-4-fluorophenyl | CH₃ | H | CH₂CH₃ | CH₂CH₃ | 124-128 |
| 34 | 4-phenoxypenyl | CH₃ | H | CH₂CH₃ | CH₂CH₃ | 125-128 |
| 35 | 4-thiomethylphenyl | CH₃ | H | CH₂CH₃ | CH₂CH₃ | 123-126 |
| 36 | 3,4-dichlorophenyl | CH₃ | H | CH₂CH₃ | CH₂CH₃ | 145-148 |
| 37 | 3-chloro-4-methoxyphenyl | CH₃ | H | CH₂CH₃ | CH₂CH₃ | 127-130 |
| 38 | 3-fluoro-4-ethoxyphenyl | CH₃ | H | CH₂CH₃ | CH₂CH₃ | 96-99 |
| 39 | 3,5-dimethylphenyl | CH₃ | H | CH₂CH₃ | CH₂CH₃ | M + H: 333 |
| 40 | 3-chloro-4-iso- | CH₃ | H | CH₂CH₃ | CH₂CH₃ | M + H: 397 |
| | propoxyphenyl | | | | | |
| 41 | 3-chloro-4-ethoxyphenyl | CH₃ | H | CH₂CH₃ | CH₂CH₃ | 116-119 |
| 42 | 2-methoxyphenyl | CH₃ | H | CH₂CH₃ | CH₂CH₃ | 97-100 |
| 43 | 3-fluorophenyl | CH₃ | H | CH₂CH₃ | CH₂CH₃ | M + H: 323 |
| 44 | 3-ethoxyphenyl | CH₃ | H | CH₂CH₃ | CH₂CH₃ | 89-92 |
| 45 | 3-fluoro-4-methoxy | CH₃ | H | CH₂CH₃ | CH₂CH₃ | 117-120 |
| 46 | 3,4-difflurophenyl | CH₃ | H | CH₂CH₃ | CH₂CH₃ | 121-124 |
| 47 | 4-ethylphenyl | CH₃ | H | CH₂CH₃ | CH₂CH₃ | 102-106 |
| 48 | 3,5-difluorophenyl | CH₃ | H | CH₂CH₃ | CH₂CH₃ | 111-113 |
| 49 | 3-isopropoxyphenyl | CH₃ | H | CH₂CH₃ | CH₂CH₃ | M + H: 363.2 |
| 50 | 2,3-difluorophenyl | CH₃ | H | CH₂CH₃ | CH₂CH₃ | 114-117 |
| 51 | 2,5-difluorophenyl | CH₃ | H | CH₂CH₃ | CH₂CH₃ | 114-118 |
| 52 | 2,5-dichlorophenyl | CH₃ | H | CH₂CH₃ | CH₂CH₃ | 117-120 |
| 53 | 3-methyl-4-fluorophenyl | CH₃ | H | CH₂CH₃ | CH₂CH₃ | 92-95 |
| 54 | 2,4-dimethoxyphenyl | CH₃ | H | CH₂CH₃ | CH₂CH₃ | M + H: 365.2 |
| 55 | 2,3-dimethylphenyl | CH₃ | H | CH₂CH₃ | CH₂CH₃ | 125-130 |
| 56 | 2,5-dimethylphenyl | CH₃ | H | CH₂CH₃ | CH₂CH₃ | M + H: 333.2 |
| 57 | 2-ethoxyphenyl | CH₃ | H | CH₂CH₃ | CH₂CH₃ | 89-96 |
| 58 | 4-trifluoromethoxyphenyl | CH₃ | H | CH₂CH₃ | CH₂CH₃ | 113-117 |
| 59 | 3,4,5-trifluorophenyl | CH₃ | H | CH₂CH₃ | CH₂CH₃ | 157-160 |
| 60 | 4-n-propoxyphenyl | CH₃ | H | CH₂CH₃ | CH₂CH₃ | 113-115 |
| 61 | 3-fluoro-4-methylphenyl | CH₃ | H | CH₂CH₃ | CH₂CH₃ | 119-122 |
| 62 | 4-iso-propoxyphenyl | CH₃ | H | CH₂CH₃ | CH₂CH₃ | 91-95 |
| 63 | 4-n-butoxyphenyl | CH₃ | H | CH₂CH₃ | CH₂CH₃ | M + H: 377.2 |
| 64 | 4-chlorophenyl | CH₃ | H | CH₂CH₂CH₂CH₂ | | 165-168 |
| 65 | 2-chlorophenyl | CH₃ | H | CH₂CH₂CH₂CH₂ | | 175-178 |
| 66 | 2-methylphenyl | CH₃ | H | CH₂CH₂CH₂CH₂ | | 156-159 |
| 67 | 2,4-difluorophenmyl | CH₃ | H | CH₂CH₂CH₂CH₂ | | 142-+145 |
| 68 | 2,4-dichlorophenyl | CH₃ | H | CH₂CH₂CH₂CH₂ | | 173-176 |
| 69 | 4-methoxyphenyl | CH₃ | H | CH₂CH₂CH₂CH₂ | | 159-163 |
| 70 | 2-methoxyphenyl | CH₃ | H | CH₂CH₂CH₂CH₂ | | 144-147 |
| 71 | 2-flurophenyl | CH₃ | H | CH₂CH₂CH₂CH₂ | | 148-152 |
| 72 | 2,3-dimethylphenyl | CH₃ | H | CH₂CH₂CH₂CH₂ | | 142-145 |
| 73 | 2-ethylphenyl | CH₃ | H | CH₂CH₂CH₂CH₂ | | 140-142 |
| 74 | 2-trifluoromethylphenyl | CH₃ | H | CH₂CH₂CH₂CH₂ | | 163-166 |
| 75 | 4-trifluoromethylphenyl | CH₃ | H | CH₂CH₂CH₂CH₂ | | 155-158 |
| 76 | 2-methyl-4-fluorophenyl | CH₃ | H | CH₂CH₃ | CH₂CH₃ | M + H: 337.3 |
| 77 | 3-methyl-4-methoxy | CH₃ | H | CH₂CH₃ | CH₂CH₃ | 101-108 |
| 78 | 3-methyl-4-chlorophenyl | CH₃ | H | CH₂CH₃ | CH₂CH₃ | 105-109 |
| 79 | thiophen-2-yl | CH₃ | H | CH₂CH₃ | CH₂CH₃ | 14-144 |
| 80 | 4-methylthiophen-3-yl | CH₃ | H | CH₂CH₃ | CH₂CH₃ | 98-102 |
| 81 | 6-fluoropyridin-3-yl | CH₃ | H | CH₂CH₃ | CH₂CH₃ | 126-129 |
| 82 | pyridin-3-yl | CH₃ | H | CH₂CH₃ | CH₂CH₃ | 93-97 |
| 83 | 6-methoxypyridin-3-yl | CH₃ | H | CH₂CH₃ | CH₂CH₃ | 113-117 |
| 84 | 2-chloropyridin-3-yl | CH₃ | H | CH₂CH₃ | CH₂CH₃ | 93-99 |
| 85 | 6-chloropyridin-3-yl | CH₃ | H | CH₂CH₃ | CH₂CH₃ | 143-147 |
| 86 | pyridin-4-yl | CH₃ | H | CH₂CH₃ | CH₂CH₃ | 127-130 |

### Test for Fungicidal Activity:

The active compounds were prepared separately or together as a stock solution with 25 mg of active compound which was made up to 10 ml using a mixture of acetone and/or dimethyl sulfoxide (DMSO) and the emulsifier Wettoll® EM 31 (wetting agent having emulsifying and dispersing action based on the ethoxylated alkylphenols) in a volume ratio of solvent/emulsifier of 99 to 1. The mixture was then made up with water to 100 ml. This stock solution was diluted with the solvent/emulsifier/water mixture described to the concentration of active compound stated below.

### Use Example 1

### Activity Against Early Blight of Tomato Caused by Alternaria solani

Leaves of potted tomato plants were sprayed to runoff point with an aqueous suspension having the active compound concentration stated below. The next day, the treated plants were inoculated with a spore suspension of Alternaria solani which contained 0.17×10⁶ spores/ml in a 2% aqueous biomalt solution. The test plants were then placed in a water vapor-saturated chamber at temperatures of from 20 to 22° C. After 5 days the disease on the untreated infected control plants had developed to such an extent that the infection of all plants could be determined visually in %.

In this test, the plants which had been treated with 250 ppm of active compound from example 4, 6, 7, 8, 9, 10, 11 or 33 showed an infection of at most 5%, whereas the untreated plants were 90% infected.

### Use Example 2

### Activity Against Gray Mold on Bell Pepper Leaves Caused by Botrytis cinerea, 1 Day Protective Application

Bell pepper leaves of the cultivar “Neusiedler Ideal Elite” were, after 2 to 3 leaves were well developed, sprayed to runoff point with an aqueous suspension having the active compound concentration stated below. The next day, the treated plants were inoculated with a spore suspension of Botrytis cinerea which contained 1.7×10⁶ spores/ml in a 2% aqueous biomalt solution. The test plants were then placed in a dark climatized chamber at 22 to 24° C. and high atmospheric humidity. After 5 days, the extent of the fungal infection on the leaves could be determined visually in %.

In this test, the plants which had been treated with 250 ppm of active compound from example 4, 6, 7, 8, 9, 10, 11, 31 or 33 showed an infection of at most 10%, whereas the untreated plants were 90% infected.

### Use Example 3

### Activity Against Net Blotch of Barley Caused by Pyrenophora teres, 1 Day Protective Application

Leaves of potted barley seedlings were sprayed to runoff point with an aqueous suspension having the active compound concentration stated below. 24 hours after the spray coating had dried on, the test plants were inoculated with an aqueous spore suspension of Pyrenophora [syn. Drechslera] teres, the net blotch pathogen. The test plants were then placed in a greenhouse at temperatures between 20 and 24° C. and 95 to 100% relative atmospheric humidity. After 6 days, the extent of the development of the disease was determined visually in % infection of the entire leaf area.

In this test, the plants which had been treated with 250 ppm of active compound from example 31 showed an infection of at most 10%, whereas the untreated plants were 90% infected.

1-19. (canceled)

20. A 3-(pyridin-2-yl)-[1,2,4]-triazine compound of the general formula I 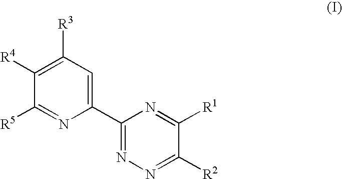
in which:
R¹, R² independently of one another are OH, halogen, NO₂, NH₂, C₁-C₈-alkyl, C₁-C₈-alkoxy, C₁-C₈-haloalkyl, C₁-C₈-haloalkoxy, C₁-C₈-alkylamino or di(C₁-C₈-alkyl)amino,
or together with the carbon atoms to which they are attached may form a saturated 5-, 6- or 7-membered carbocycle or heterocycle which, in addition to the carbon ring members, has one or two heteroatoms selected from the group consisting of oxygen and sulfur as ring members, where the carbocycle and the heterocycle are unsubstituted or have 1, 2, 3 or 4 C₁-C₄-alkyl groups as substituents;
R³ is hydrogen, C₁-C₄-alkyl, C₁-C₄-alkoxy, C₁-C₄-haloalkyl, C₁-C₄-haloalkoxy, C₃-C₆-cycloalkyl, C₃-C₆-cycloalkylmethyl, or halogen;
R⁴ is C₁-C₄-alkyl, C₁-C₄-alkoxy, C₁-C₄-haloalkyl, C₁-C₄-haloalkoxy or halogen;
R⁵ is hydrogen, C₁-C₈-alkyl, C₁-C₈-haloalkyl, C₁-C₈-alkoxy, C₁-C₈-haloalkoxy, C₃-C₈-cycloalkyl, C₃-C₈-cycloalkyloxy, 5- or 6-membered heteroaryl, phenyl, phenoxy, benzyl, benzyloxy, 5- or 6-membered heteroarylmethyl or 5- or 6-membered heteroaryloxy, where the cyclic radicals mentioned above are unsubstituted or may have 1, 2, 3, 4 or 5 radicals R^{a}, where
R^{a} is selected from the group consisting of OH, SH, halogen, NO₂, NH₂, CN, COOH, C₁-C₈-alkyl, C₁-C₈-alkoxy, C₁-C₈-haloalkyl, C₁-C₈-haloalkoxy, C₁-C₈-alkylamino, di(C₁-C₈-alkyl)amino, C₁-C₈-alkylthio, C₁-C₈-haloalkylthio, C₁-C₈-alkylsulfinyl, C₁-C₈-haloalkylsulfinyl, C₁-C₈-alkylsulfonyl, C₁-C₈-haloalkylsulfonyl, C₃-C₈-cycloalkyl, phenyl, phenoxy and radicals of the formula C(=Z)R^{aa} in which Z is O, S, N(C₁-C₈-alkyl), N(C₁-C₈-alkoxy), N(C₃-C₈-alkenyloxy) or N(C₃-C₈-alkynyloxy) and R^{aa} is hydrogen, C₁-C₈-alkyl, C₁-C₈-alkoxy, NH₂, C₁-C₈-alkylamino or di(C₁-C₈-alkyl)amino, or two radicals R^{a} attached to adjacent carbon atoms together with the carbon atoms to which they are attached may also form a saturated 5-, 6- or 7-membered carbocycle, a benzene ring or a 5-, 6- or 7-membered heterocycle which, in addition to the carbon ring members, has one or two heteroatoms selected from the group consisting of oxygen and sulfur as ring members, where the carbocycle and the heterocycle are unsubstituted or have 1, 2, 3 or 4 C₁-C₄-alkyl groups as substituents;
or an agriculturally useful salt of a compound of the formula I, except for:
2,6-bis-(5,6-dimethyl-1,2,4-triazine-3-yl)pyridine;
2,6-bis-(5,6-diethyl-1,2,4-triazine-3-yl)pyridine;
2,6-bis-(5,6-dipropyl-1,2,4-triazine-3-yl)pyridine;
2,6-bis-(5,6-diisopropyl-1,2,4-triazine-3-yl)pyridine;
2,6-bis-(5,6-dibutyl-1,2,4-triazine-3-yl)pyridine;
2,6-bis-(5,6-diisobutyl-1,2,4-triazine-3-yl)pyridine;
2,6-bis-(5,6-dipentyl-1,2,4-triazine-3-yl)pyridine;
2,6-bis-(5,6-dihexyl-1,2,4-triazine-3-yl)pyridine;
2,6-bis-(5,6-diheptyl-1,2,4-triazine-3-yl)pyridine;
3-[6-(2,2′-bipyridyl)]-5,6-dimethyl-1,2,4-triazine;
3-[6-(2,2′-bipyridyl)]-5,6-diethyl-1,2,4-triazine;
3-[6-(2,2′-bipyridyl)]-5,6-dipropyl-1,2,4-triazine;
3-[6-(2,2′-bipyridyl)]-5,6-dibutyl-1,2,4-triazine;
5,6-diethyl-3-[6-(2-pyridyl)-4-methoxypyridine-2-yl]-1,2,4-triazine;
3-[6-methylpyridine-2-yl)-5,6-dimethyl-1,2,4-triazine;
3-[6-methylpyridine-2-yl)-5,6-diethyl-1,2,4-triazine;
2,6-bis-(5,6-dimethoxy-1,2,4-triazine-3-yl)pyridine; and
2,6-bis-(5,6-diethoxy-1,2,4-triazine-3-yl)pyridine.

21. The compound according to claim 20 in which R¹ and R² independently of one another are selected from the group consisting of fluorine, chlorine, C₁-C₄-alkyl, methoxy, ethoxy, CF₃, CHF₂, OCF₃ and OCHF₂.

22. The compound according to claim 20 in which R¹ and R² together with the carbon atoms of the triazine ring to which they are attached are one of the following rings: 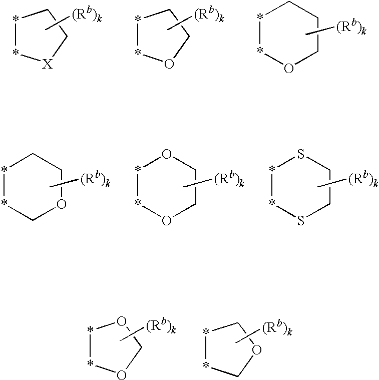
in which
* are the atoms of the triazine ring;
k is 0, 1, 2, 3 or 4;
R^{b} is C₁-C₄-alkyl; and
X is (CH₂)ₙ where n=1, 2 or 3 and where 1, 2, 3 or 4 of the hydrogen atoms in (CH₂)ₙ may be replaced by R^{b} if k≠0.

23. The compound according to claim 20 in which R¹ and R² are C₁-C₄-alkyl or together with the carbon atoms of the triazine ring to which they are attached are a ring of the formula 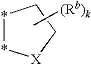
in which
* are the atoms of the triazine ring;
k is 0, 1, 2, 3 or 4;
R^{b} is C₁-C₄-alkyl; and
X is (CH₂)ₙ where n=2 or 3 and where 1, 2, 3 or 4 of the hydrogen atoms in (CH₂)ₙ may be replaced by R^{b} if k≠0.

24. The compound according to claim 20 in which R³ is hydrogen, fluorine, chlorine, C₁-C₄-alkyl, methoxy, ethoxy, CF₃, CHF₂, OCF₃ or OCHF₂.

25. The compound according to claim 24 in which R³ is hydrogen.

26. The compound according to claim 24 in which R³ is chlorine, CF₃, methyl or methoxy.

27. The compound according to claim 20 in which R⁴ is hydrogen, fluorine, chlorine, methyl, ethyl, methoxy, ethoxy, CF₃, CHF₂, OCF₃ or OCHF₂.

28. The compound according to claim 27 in which R⁴ is hydrogen, fluorine, chlorine or methyl.

29. The compound according to claim 20 in which R^{a} is selected from the group consisting of halogen, C₁-C₄-alkyl, C₁-C₄-alkoxy, C₁-C₄-alkylcarbonyl, C₁-C₄-alkoxycarbonyl, and radicals of the formula C(═N—O—C₁-C₈-alkyl)R^{aa} in which R^{aa} is hydrogen or C₁-C₄-alkyl.

30. The compound according to claim 20 in which R⁵ is phenyl, phenoxy or benzyl in which the phenyl ring has 1, 2, 3, 4 or 5 radicals R^{a}.

31. The compound according to claim 30 in which the phenyl ring in phenyl, phenoxy or benzyl has the general formula P 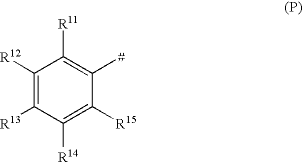
in which # is the point of attachment to the remainder of the molecule;
R¹¹ is hydrogen, fluorine, chlorine, CH₃, OCH₃, OCHF₂, OCF₃ or CF₃;
R¹², R¹⁴ independently of one another are hydrogen, chlorine, fluorine, CH₃, OCH₃, OCHF₂, OCF₃ or CF₃, where one of the radicals R¹² and R¹⁴ may also be NO₂, C(O)CH₃ or COOCH₃;
R¹³ is hydrogen, fluorine, chlorine, cyano, OH, CHO, NO₂, NH₂, methylamino, dimethylamino, diethylamino, C₁-C₄-alkyl, C₃-C₈-Cycloalkyl, C₁-C₄-alkoxy, C₁-C₄-alkylthio, C₁-C₄-haloalkyl, C₁-C₄-haloalkoxy, CO(A²), in which A² is C₁-C₄-alkyl or C₁-C₄-alkoxy, or a group C(R^{13a})═NOR^{13b} in which R^{13a} is hydrogen or methyl and R^{13b} is C₁-C₄-alkyl, propargyl or allyl, or R¹² and R¹³ together form a group O—CH₂—O; and
R¹⁵ is hydrogen, fluorine, chlorine or C₁-C₄-alkyl.

32. The compound according to claim 20 in which R⁵ is C₁-C₆-alkyl or C₁-C₆-haloalkyl.

33. The compound according to claim 20 in which R⁵ is selected from the group consisting of 5-membered heteroaryl which has 1, 2, 3 or 4 nitrogen atoms or I heteroatom selected from the group consisting of oxygen and sulfur and optionally 1, 2 or 3 nitrogen atoms as ring atoms and 6-membered hetaryl which has 1, 2, 3 or 4 nitrogen atoms as ring members, where 5- and 6-membered hetaryl may have 1, 2, 3 or 4 substituents R^{a}.

34. The compound according to claim 33 in which R⁵ is selected from the group consisting of furyl, thienyl, pyridinyl and pyrimidinyl which are in each case unsubstituted or have 1, 2 or 3 substituents R^{a}.

35. The use of a compound of the formula I according to claim 20 or of a salt thereof for controlling phytopathogenic fungi.

36. A crop protection composition comprising a solid or liquid carrier and a compound of the formula I according to claim 20 and/or a salt thereof.

37. Seed comprising at least one compound of the formula I according to claim 20 and/or a salt thereof.

38. A method for controlling phytopathogenic fungi wherein the fungi, or the materials, plants, the soil or seed to be protected against fungal attack are/is treated with an effective amount of a compound of the formula I according to claim 20 or a salt thereof.
